(19) United States
(12) Patent Application Publication
(10) Pub. No.: US 2013/0266939 A1
(43) Pub. Date: Oct. 10, 2013

(54) SYSTEMS AND METHODS FOR STUDYING INFLAMMATION-DRUG INTERACTIONS

(71) Applicant: HEPREGEN CORPORATION, Medford, MA (US)

(72) Inventors: Michael McVay, Boston, MA (US); Chitra Kanchagar, Auburn, MA (US); Okechukwu Ukairo, Marlborough, MA (US); Salman Khetani, Fort Collins, CO (US)

(73) Assignee: HEPREGEN CORPORATION, Medford, MA (US)

(21) Appl. No.: 13/797,002

(22) Filed: Mar. 12, 2013

Related U.S. Application Data
(60) Provisional application No. 61/609,732, filed on Mar. 12, 2012.
(60) Provisional application No. 61/709,020, filed on Oct. 2, 2012.
(60) Provisional application No. 61/713,804, filed on Oct. 15, 2012.

(51) Int. Cl. G01N 33/50; C12Q 1/68; C12Q 1/26; C12N 5/071

(52) U.S. Cl. CPC G01N 33/5088; C12N 5/0697; C12Q 1/6809; C12Q 1/26
USPC 435/6.11; 435/347; 435/305.1; 435/289.1; 435/373; 435/6.13; 435/34; 435/25

(57) ABSTRACT

The present disclosure provides compositions, systems, and tools for modeling liver inflammation and methods of using the same. The disclosure provides micropatterned hepatocyte co-cultures where individual cell populations remain functionally stable during long-term culture. The in vitro liver inflammation models of the present disclosure may be useful for evaluating inflammation-mediated toxicities of compounds in a pre-clinical setting.

### RELATED APPLICATIONS

This application claims the benefit of U.S. Provisional Application No. 61/609,732 filed Mar. 12, 2012, U.S. Provisional Application No. 61/709,020 filed Oct. 2, 2012, and U.S. Provisional Application No. 61/713,804 filed Oct. 15, 2012, the disclosures of which are incorporated herein by reference in their entirety.

### BACKGROUND OF THE INVENTION

Liver failure is the cause of death of over 30,000 patients in the United States every year and over 2 million patients worldwide. Drug-induced liver disease is a major challenge for the pharmaceutical industry since unforeseen liver toxicity causes many new drug candidates to fail either in clinical trials or after release. In vitro cell culture techniques can be used to study human hepatic tissue cells, and the effects of various drugs on the cells. Additionally, in vitro models can provide valuable information on drug uptake and metabolism, enzyme induction, and drug interactions affecting metabolism and hepatotoxicity. However, human hepatic tissue cells are difficult to maintain in culture as they rapidly lose viability and phenotypic functions.

### SUMMARY OF THE INVENTION

In certain aspects, the disclosure provides a micropatterned co-culture comprising: (a) a population of hepatocytes defining a cellular island, wherein the cellular island comprises a diameter or width of about 250 μm to 750 μm; (b) a population of stromal cells, wherein the stromal cells define a geometric border of the cellular island; and (c) a population of Kupffer cells, wherein the micropatterned co-culture of hepatocytes and Kupffer cells maintains long-term functional stability.

In certain aspects, the disclosure provides a method for producing a micropatterned co-culture containing at least three cell types, the method comprising: (a) spotting an adherence material on a substrate at spatially different locations, each spot having a defined geometric pattern, wherein the defined geometric pattern comprises a diameter or width of about 250 μm to 750 μm; (b) contacting the substrate with a population of hepatocytes that selectively adhere to the adherence material and/or substrate; (c) culturing the hepatocytes on the substrates to generate a plurality of cellular islands; and (d) contacting the substrate with a stromal cell population that adheres to the substrate at a location different than the hepatocyte population, wherein the cells of the stromal cell population define a geometric border of the cellular island, to generate a hepatocyte-stromal cell co-culture; (e) maintaining the hepatocyte-stromal cell co-culture for a period of time sufficient to allow the hepatocytes to functionally stabilize; and (f) contacting the hepatocytes and stromal cells with a population of Kupffer cells; wherein the micropatterned co-culture of hepatocytes and the Kupffer cells maintains long-term functional stability.

In certain aspects, the disclosure provides a cellular composition made by the method disclosed herein.

In certain aspects, the disclosure provides a method of determining the interaction of one or more test compounds with hepatocytes comprising: a) contacting a micropatterned co-culture comprising hepatocytes, stromal cells, and Kupffer cells with one or more test agents; and b) measuring a characteristic of the one or more test compounds or an activity of the hepatocytes, wherein the characteristic or activity measured in (b) indicates the interaction of one or more test compounds with hepatocytes and wherein the micropatterned co-culture of hepatocytes and Kupffer cells maintains long-term functional stability.

In certain aspects, the disclosure provides a method of determining the effect of liver inflammation on one or more test compounds comprising: a) contacting a micropatterned co-culture comprising hepatocytes, stromal cells, and Kupffer cells with an inflammation-inducing agent to generate an in vitro model of liver inflammation; b) contacting the in vitro model of liver inflammation generated in step (a) with one or more test agents; and c) measuring a characteristic of the one or more test compounds or an activity of the hepatocytes, wherein the characteristic or activity measured in (c) indicates the effect of liver inflammation on the one or more test compounds. In some embodiments, the method further comprises: d) contacting a second micropatterned co-culture comprising hepatocytes, stromal cells, and Kupffer cells with the one or more test agents; e) measuring the activity selected in (b) of the second co-culture hepatocytes; and f) comparing the measurements in step (b) and (e).

In certain aspects, the disclosure provides a method of determining inflammation-mediated toxicity of a test agent, comprising; a) contacting a first micropatterned co-culture comprising hepatocytes, stromal cells, and Kupffer cells with the test agent; b) measuring an activity selected from gene expression, cell function, metabolic activity, morphology, and a combination thereof, of the hepatocytes; c) contacting a second micropatterned co-culture comprising hepatocytes, stromal cells, and Kupffer cells with an inflammation-inducing agent to generate an in vitro model of liver inflammation; d) contacting the in vitro model of liver inflammation generated in step (c) with the test agent; e) measuring the activity selected in (b) of the second co-culture hepatocytes; and f) comparing the measurements in step (b) and (e), to determine the inflammation-mediated toxicity of the test agent. In certain embodiments, steps (a)-(b) are replaced by a standard measurement for comparison in step (f).

In some embodiments, the stromal cells are fibroblast cells or fibroblast derived cell.

In some embodiments, the micropatterned co-culture further comprises one or more populations of non-parenchymal cells. In some embodiments, the one or more populations of non-parenchymal cells are selected from the group consisting of Ito cells, endothelial cells, biliary duct cells, immune-mediating cells, and stem cells. In some embodiments, the immune-mediating cells are selected from the group consisting of macrophages, T cells, neutrophils, dendritic cells, mast cells, eosinophils and basophils. In some embodiments, the co-culture does not contain any additional cell types.

In some embodiments, the ratio of hepatocytes to Kupffer cells in the micro-patterned co-culture is 1:0.1. In some embodiments, the ratio of hepatocytes to Kupffer cells in the micro-patterned co-culture is 1:0.4.

In some embodiments, the cellular islands are spaced apart from about 1200 μm to 1300 μm from center to center of the cellular islands.

In some embodiments, the micropatterned co-culture is located in a microfluidic device. In some embodiments, the micropatterned co-culture is located in a tissue culture plate.

In some embodiments, the micropatterned co-culture of hepatocytes and Kupffer cells maintains long-term functional stability for at least 10 days.

In some embodiments, the hepatocytes and Kupffer cells are selected from the group consisting of human cells, rat cells, mouse cells, monkey cells, dog cells, fish cells and guinea pig cells.

In some embodiments, the time sufficient to allow the hepatocytes to functionally stabilize is at least 7 days.

In some embodiments, the functional stability of the heptocytes is determined by measuring an activity selected from gene expression, cell function, metabolic activity, morphology, and a combination thereof, of the hepatocytes.

In some embodiments, the metabolic activity is selected from CYP3A4 activity, urea synthesis, and albumin secretion.

In some embodiments, the activity of the hepatocytes is selected from gene expression, cell function, metabolic activity, morphology, cytokine secretion, protein or metabolite secretion, and a combination thereof.

In some embodiments, the ratio of the Kupffer cells to the hepatocytes corresponds to the ratio of the cells in an inflamed state of the liver. In some embodiments, the ratio of the Kupffer cells to the hepatocytes corresponds to the ratio of the cells in a physiologically normal state of the liver.

In some embodiments, the test agent is selected from the group consisting of a cytotoxic agent, pharmaceutical agent, a small molecule, and a xenobiotic.

In some embodiments, the metabolic activity is protein production. In some embodiments, the metabolic activity is enzyme bioproduct formation. In some embodiments, the metabolic activity is a CYP450 isoenzyme activity. In some embodiments, the CYP450 isoenzyme is selected from the group consisting of CYP1A2, CYP1B1, CYP2A6, CYP2B6, CYP2C, CYP2D6, CYP2E1, CYP2F1, CYP2J2, CYP3A4, CYP4A, and CYP4B.

In some embodiments, the methods disclosed herein are used in determining inflammation-mediated toxicity of a test agent. In some embodiments, the methods disclosed herein are used in determining inflammation-mediated effects on co-administered test agent combinations.

In some embodiments, a characteristic of the one or more test compounds to be measured is selected from mass, structure, quantity and a combination thereof.

In some embodiments, the inflammation-inducing agent is LPS. In some embodiments, the inflammation-inducing agent is IL-1B. In some embodiments, the inflammation-inducing agent is selected from the group consisting of a cytotoxic agent, pharmaceutical agent, and a xenobiotic.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the HepatoPac™ platform. Long-term functional stability of Human HepatoPac™ cultures, demonstrated by Phase I and Phase II drug metabolism activity, is also shown.

FIGS. 2A and 2B show CYP450 3A4 activity and urea synthesis in hepatocytes from two donors at two days, six days, and ten days after Kupffer cell addition to the co-culture. HP=HepatoPac™ alone, HP:KC=HepatoPac™/Kupffer cell co-cultures at various ratios.

FIGS. 3A-3C show phagocytic activity of Kupffer cells in co-culture. FIG. 3D shows immuno-staining of Kupffer cells at Day 10 of co-culture with anti-CD68 in green.

FIGS. 4A and 4B show the effect of LPS at Days 1, 2, 3, and 4 post addition of exposure on HepatoPac™-Kupffer cell co-cultures, demonstrated by IL-6 levels. FIG. 4C shows the effect of LPS stimulation on the cellular ATP content of HepatoPac™-Kupffer cell co-cultures.

FIGS. 5A and 5B show the effect of cytokine exposure on HepatoPac™-Kupffer cell co-cultures, demonstrated by IL-6 levels.

FIGS. 6A and 6B show cytokine-mediated inhibition of Cyp450 3A4 activity in HepatoPac™-Kupffer cell co-cultures.

FIGS. 7A-7D show cytokine-mediated repression of cytochrome expression in HepatoPac™-Kupffer cell co-cultures.

FIGS. 8A and 8B show the effect of cytokine exposure on cell viability in HepatoPac™-Kupffer cell co-cultures, demonstrated by ATP levels.

FIGS. 9A-9B show the characterization of rat HepatoPac™-Kupffer cell co-cultures. FIG. 9A is a graph of CYP450 3A4 Glo activity, which shows the variation in percent control at each of one, three and five days in culture after LPS stimulation. FIG. 9B shows TNF-α secretion, at each of one, three and five days after LPS stimulation.

FIGS. 10A-10C show the effect of LPS treatment on trovafloxacin (TVX) toxicity in rat HepatoPac™-Kupffer cell co-cultures. FIGS. 10A and 10B show cellular ATP content of the various co-cultures and illustrate variation in percent control versus Trovafloxacin (Cmax) and Levofloxacin (Cmax), respectively. FIG. 10C summarizes the observed TVX TC50 values.

FIGS. 11A-11C show the effect of treatment with pentoxifylline (an inhibitor of TNF-α transcription) on TVX/LPS-induced HepatoPac™ toxicity and TNF-α secretion.

FIGS. 12A-12F show the effect of LPS or TNF-α treatment on trovafloxacin (TVX) toxicity in human HepatoPac™-Kupffer cell co-cultures. FIG. 12A shows cytokine secretion by Kupffer cell-only cultures. FIG. 12B shows IL-6 secretion versus Trovafloxacin (Cmax) in HepatoPac™ or Kupffer-cell only cultures. FIGS. 12C and D show cellular ATP content of the various co-cultures and illustrate variation in percent control versus Trovafloxacin (Cmax) and Levofloxacin (Cmax), respectively. FIG. 12E shows the effect of TNF-α addition on TVX toxicity. FIG. 12F summarizes the observed TVX TC50 values.

### DETAILED DESCRIPTION

The present disclosure provides compositions, systems, and tools for modeling the liver and methods of using the same.

### (i) OVERVIEW

Historically cell culture techniques and tissue development failed to take into account the necessary microenvironment for cell-cell and cell-matrix communication as well as an adequate diffusional environment for delivery of nutrients and removal of waste products. Cell culture techniques and understanding of the complex interactions cells have with one another and the surrounding environment have improved in the past decade.

While many methods and bioreactors have been developed to grow tissue for the purposes of generating artificial tissues for transplantation or for toxicology studies, these bioreactors do not adequately simulate, in vitro, the mechanisms by which nutrients, gases, and cell-cell interactions are delivered and performed in vivo. For example, cells in living tissue are “polarized” with respect to diffusion gradients. Differential delivery of oxygen and nutrients, as occurs in vivo by means of the capillary system, controls the relative functions of tissue cells and their maturation. Thus, cell culture systems and bioreactors that do not simulate these in vivo delivery mechanisms do not provide a sufficient corollary to in vivo environments to develop tissues or measure tissue responses in vitro.

Drug-induced liver disease represents a major economic challenge for the pharmaceutical industry since unforeseen liver toxicity and poor bioavailability issues cause more than 50% of new drug candidates to fail in Phase I clinical trials. Also, a third of drug withdrawals from the market and more than half of all warning labels on approved drugs are primarily due to adverse affects on the liver. Therefore, besides pharmacological properties, ADME/Tox (absorption, distribution, metabolism, excretion and toxicity) characteristics are crucial determinants of the ultimate clinical success of a drug. This realization has led to an early introduction of ADME/Tox screening during the drug discovery process, in an effort to select against drugs with problematic properties.

Animal models provide a limited view of human toxicity due to species-specific variations as well as animal-to-animal variability, necessitating 5-10 animals per compound per dose, sometimes in both genders. Incorporating in vitro models into drug development provides several advantages: earlier elimination of problematic drugs, reduction in variability by allowing hundreds of experiments per animal and human models without patient exposure. In the case of the liver, in vitro models can provide valuable information on drug uptake and metabolism, enzyme induction, and drug-drug interactions affecting metabolism and hepatotoxicity.

Several in vitro liver models are used for short-term (hours) investigation of xenobiotic metabolism and toxicity. Perfused whole organs, liver slices and wedge biopsies maintain many aspects of liver's in vivo microenvironment; however, such systems suffer from limited drug availability to inner cell layers, limited viability (<24 h) and are not suitable for enzyme induction studies. Isolated liver microsomes, which are cellular fragments that contain mostly CYP450 enzymes, are used primarily to investigate drug metabolism via the phase I pathways (oxidation, reduction, hydrolysis and the like). However, microsomes lack many important aspects of the cellular machinery where dynamic changes occur (i.e. gene expression, protein synthesis) to alter drug metabolism, toxicity and drug-drug interactions. Besides microsomes, cell lines derived from hepatoblastomas (HepG2) or from immortalization of primary hepatocytes (HepLiu, SV40 immortalized) are finding limited use as reproducible, inexpensive models of hepatic tissue. However, no cell line has been developed to date that maintains physiologic levels of liver-specific functions. Usually such cell lines are plagued by an abnormal repertoire of hepatic functions.

Current in vitro liver models used by the pharmaceutical industry, though useful in a limited capacity, are not fully predictive of in vivo liver metabolism and toxicity. Thus, research has increasingly turned towards using isolated primary human hepatocytes as the gold standard for in vitro studies; however, hepatocytes are notoriously difficult to maintain in culture as they rapidly lose viability and phenotypic functions.

Moreover, cases of idiosyncratic toxicity are often mediated by the occurrence of an episode of inflammatory stress. For example, the appearance or relief of inflammation through drug therapy (i.e. therapeutic proteins) may differentially affect levels of enzymes involved in metabolism of co-administered drugs with potential pharmacological and toxicological consequences. An in vitro model that mimics liver inflammation may provide better predictive data in preclinical testing.

The invention provides methods, tools, and compositions that overcome the limitations of current techniques. The disclosure provides stable micropatterned hepatocyte tri-cultures for modeling the normal as well as inflamed liver states in vitro. The co-cultures of the invention have distinct advantages over current in-vitro 3-D model liver systems in terms of simplicity, ease of use, adaptability and scalability for high-throughput applications. In addition, the individual cell populations of the co-culture maintain functional stability during long-term culturing. This unexpected property facilitates the implementation and development of assays, such as long-term evaluation of drug toxicity profiles, which were not feasible earlier due to limited cell functionality.

### (ii) DEFINITIONS

For convenience, certain terms employed in the specification, examples, and appended claims are collected here.

As used herein, the singular forms “a,” “and,” and “the” include plural referents unless the context clearly dictates otherwise. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

As used herein, the term “micropattern” refers to a pattern formed on a substrate (e.g., by a protein, cell, or combination of cells of two or more types), which has a spatial resolution (e.g., 1-5 μm) that permits spatially controlling cell placement at the single-cell level. Thus, using micropatterning methods, one can precisely manipulate cell-cell interactions.

A cellular island or “spot” refers to a bounded geometrically defined shape of a substantially homogenous cell-type having a defined border. In one aspect, the cellular island or spot is surrounded by different cell-types, materials (e.g., extracellular matrix materials) and the like. The cellular islands can range in size and shape (e.g., may be of uniform dimensions or non-uniform dimensions). Cellular islands may be of different shapes on the same substrate. Furthermore, the distance between two or more cellular islands can be designed using methods known in the art (e.g., lithographic methods and spotting techniques). The distances between cellular islands can be random, regular or irregular. The distance between and/or size of the cellular islands can be modified to provide a desired phenotypic characteristic of morphology to a particular cell types (e.g., a parenchymal cell such as a hepatocyte).

### (iii) MICROPATTERNED HEPATOCYTE CO-CULTURES

The invention augments micropatterned hepatocyte-stromal cell co-cultures by the addition of one or more populations of non-parenchymal cells. The micropatterned configurations (from single cellular islands to large aggregates) outperform randomly distributed co-cultures. Amongst the micropatterned configurations that were engineered, a balance of homotypic and heterotypic interactions can yield functional co-cultures having defined or desired phenotypic activity, longevity and proliferative capacity.

The morphology and function of cells in an organism vary with respect to their environment, including distance from sources of metabolites and oxygen as well as homotypic and heterotypic cell interactions. For example, the morphology and function of hepatocytes are known to vary with position along the liver sinusoids from the portal triad to the central vein (Bhatia et al., Cellular Engineering 1:125-135, 1996; Gebhardt R. Pharmaol Ther. 53 (3):275-354, 1992; Jungermann K. Diabete Metab. 18 (1):81-86, 1992; and Lindros, K. O. Gen Pharmacol. 28 (2):191-6, 1997). This phenomenon, referred to a zonation, has been described in virtually all areas of liver function. Oxidative energy metabolism, carbohydrate metabolism, lipid metabolism, nitrogen metabolism, bile conjugation, and xenobiotic metabolism, have all been localized to separate zones. Such compartmentalization of gene expression is thought to underlie the liver's ability to operate as a ‘glucostat’ as well as the pattern of zonal hepatotoxicity observed with some xenobiotics (e.g., environmental toxins, chemical/biological warfare agents, natural compounds such as holistic therapies and nutraceuticals).

Isolated human parenchymal cells (such as hepatocytes) are highly unstable in culture and are therefore of limited utility for studies on drug toxicity, drug-drug interaction, drug-related induction of detoxification enzymes, and other phenomena. In spite of their recognized advantages, primary parenchymal cells are notoriously difficult to maintain in culture as they rapidly lose viability and phenotypic functions upon isolation from their in vivo microenvironment. Isolated hepatocytes rapidly lose important liver-specific functions such as albumin secretion, urea synthesis and cytochrome P450 activity. After about a week in culture on collagen-coated dishes, hepatocytes show a fibroblastic morphology. Freshly isolated hepatocytes, on the other hand, show a polygonal morphology with distinct nuclei and nucleoli and bright intercellular boundaries (bile canaliculi). De-differentiated hepatocytes are typically unresponsive to enzyme inducers, which severely limits their use.

Over the last couple of decades, investigators have been able to stabilize several hepatocyte functions using soluble factor supplementation, extracellular matrix manipulation, and random co-culture with various liver and non-liver derived stromal cell types. Addition of low concentrations of hormones, corticosteroids, cytokines, vitamins, or amino acids can help stabilize liver-specific functions in hepatocytes. Presentation of extracellular matrices of different composition and topologies can also induce similar stabilization. For instance, hepatocytes from a variety of species (human, mouse, rat) secrete albumin when sandwiched between two layers of rat tail collagen-I (double-gel). However, studies have shown that CYP450 activities decline in the double-gel model, and the presence of an overlaid layer of collagen presents transport barriers for drug candidates, thus limiting their use as assay systems. Culture on a tumor-derived basement membrane extract called Matrigel also induces hepatocyte spheroid formation and leads to retention of key hepatocyte functions including P450 activity. While Matrigel can induce functions in rodent hepatocytes, it appears to have fewer effects on human hepatocytes. Though they may find use in specific scenarios during drug discovery and development, most in vitro liver models in use have limited applicability to the development of a robust biomimetic liver platform. For instance, defined media formulations limit the contents of the perfusate, sandwich culture adds a transport barrier and hepatocytes do not express gap junctions, and Matrigel and spheroid culture rely on hepatocyte aggregation with resultant non-uniformity and transport barriers.

The invention overcomes many of these problems by optimizing the homotypic and heterotypic interactions of parenchymal cells with non-parenchymal cells. For example, in the adult liver, hepatocytes interact with a variety of non-parenchymal cell types including sinusoidal endothelia, stellate cells, Kupffer cells and fat-storing Ito cells (e.g., heterotypic interactions). These non-parenchymal cell types modulate cell fate processes of hepatocytes under both physiologic and pathophysiologic conditions. In vitro, random co-cultivation of primary hepatocytes with a plethora of distinct non-parenchymal cell types from different species and organs has been shown to support differentiated hepatocyte function for several weeks in a manner reminiscent of hepatic organogenesis. These random hepatocyte co-cultures have been used to study various aspects of liver physiology and pathophysiology such as lipid metabolism, and induction of the acute-phase response.

The liver contains several resident cell types in addition to hepatocytes, including stellate cells, cholangiocytes, oval cells, Kupffer cells, and sinusoidal endothelial cells. In the adult liver, the majority of liver cells are hepatocytes, with stellate cells and cholangiocytes representing minority populations of cells. Stellate cells function as the primary source of extracellular matrix in normal and diseased liver, transitioning from a quiescent vitamin-A rich cell to a highly fibrogenic cell during activation caused by liver injury. Cholangiocytes line the intrahepatic biliary tree inside the liver. Cholangiocytes play a key role in the modification of bile, secreted by hepatocytes, by a series of reabsorbtive and secretory processes under both spontaneous and hormone-regulated conditions. Cholangiocytes also have the ability to selectively proliferate during injury such as bile duct ligation. Oval cells are found in the periportal region of the liver under some conditions, and have been postulated to function as a bi-potential precursor cell with the ability to give rise to hepatocytes and cholangiocytes (also known as bile duct cells).

An exemplary micropatterned bi-culture is the HepatoPac™, which provides, in a multi-well format (up to 96-well), in vitro models of human and animal (i.e. rat, dog, monkey) livers (Khetani and Bhatia, Nat Biotechnol. 26(1):120-126, 2007). Primary hepatocytes are organized into colonies of prescribed, empirically-optimized dimensions and subsequently surrounded by supportive stromal cells. Hepatocytes in HepatoPac™ retain their in vivo-like morphology, express a complete complement of liver-specific genes, metabolize compounds using active Phase I/II drug metabolism enzymes, secrete diverse liver-specific products, and display functional bile canaliculi for 4-6 weeks in vitro (Wang et al. Drug Metab Dispos. 38(10):1900-1905, 2010). The balance of homotypic and heterotypic interactions between hepatocytes and stromal cells is very important for the long-term functional stability of hepatocytes in bi-culture. Therefore, it was not known whether addition of other cell types, such as Kupffer cells, to generate higher order co-cultures would still allow the individual cell types to maintain long-term functional stability. Herein, we demonstrate that addition of Kupffer cells do not compromise hepatic functionality, and both the hepatocytes and the Kupffer cells remained functional during long-term culturing.

Isolated primary human hepatocytes in adherent culture are widely considered to be the most suitable for in vitro testing (Hewitt et al. Drug Metab Rev. 39(1):159-234, 2007). The invention provides micropatterned cultures comprising cellular islands of parenchymal cells such as heptocytes, surrounded by stromal cells, and one or more populations of non-parenchymal cells. Microtechnology tools are used to both optimize and miniaturize in vitro models of human and animal livers. The micropatterned co-cultures are able to maintain functional stability during long-term culture. In this aspect, a substrate is modified and prepared such that the stromal cells and non-parenchymal cells are interspersed with islands of parenchymal cells, such as heptocytes. Using microfabrication techniques modified, for example, from the semiconductor industry, the substrate is modified to provide for spatially arranging parenchymal cells (e.g., human hepatocytes) and supportive stromal cells (e.g., fibroblasts) and one or more populations of non-parenchymal cells in a miniaturizable format. The spatial arrangements can be a parenchymal cell type comprising a bounded geometric shape. The bounded geometric shape can be any shape (e.g., regular or irregular) having dimensions defined by the shape (e.g., diameter, width, length and the like). The dimensions will have a defined scale based upon their shape such that at least one distance from one side to a substantially opposite side is about 200-800 μm (e.g., where the shape is rectangular or oval, the distance between one side to an opposite side is 200-800 μm). The cellular islands may be spaced apart 1200 μm to 1300 μm from center to center of the cellular islands. For example, parenchymal cells (e.g., hepatocytes) can be prepared in circular islands of varying dimensions (e.g., 36 μm, 100 μm, 490 μm, 4.8 mm, and 12.6 mm in diameter; typically about 250-750 μm with about 1200 μm spacing) surrounded by stromal cells (e.g., fibroblast such as murine 3T3 fibroblasts) and one or more populations of non-parenchymal cells (e.g., Kupffer cells). For example, hepatocyte detoxification functions are maximized at small patterns, synthetic ability at intermediate dimensions, while metabolic function and normal morphology were retained in all patterns.

In certain embodiments, a micropatterned bi-culture comprising cellular islands of primary hepatocytes surrounded by stromal cells that define the geometric border of the cellular islands is first allowed to functionally stabilize prior to the addition of one or more populations of non-parenchymal cells. This is particularly important when hepatocytes in culture are derived from sources such as, for example, cryopreserved hepatocytes. It is hypothesized that when such cells are grown in culture, there is an initial growth phase during which liver-specific functions steadily improve until they reach steady-state levels. Functional stability of the hepatocytes is determined by measuring liver-specific functions such as, but not limited to, liver-specific functions such as albumin secretion, urea synthesis and cytochrome P450 activity. Various liver-specific metabolic assays and transporter assays are known in the art and can be employed to evaluate the functional stability of hepatocytes in culture. In some embodiments, the functional stability is measured against values determined for freshly isolated hepatocytes. In some embodiments, the functional stability is measured against values determined for a double gel culture. In some embodiments, the cut-off for determining functional stability of the hepatocytes in culture is at least 50%, at least 60%, at least 70%, or at least 80% of the values determined for freshly isolated hepatocytes. It will be appreciated by one of skill in the art that there will be species- and donor-specific differences in the activity levels. There will be species- and donor-specific differences in the time period it takes for the hepatocytes to functionally stabilize as well. In some embodiments, it takes up to 7 days for the hepatocytes to functionally stabilize in the bi-culture. In some embodiments, it takes 4-7, 5-8, 6-9 or 7-10 days for the hepatocytes to functionally stabilize in the bi-culture. In some embodiments, the micropatterned bi-culture is a co-culture of primary human hepatocytes and embryonic fibroblasts (HepatoPac™) that retains high levels of phenotypic functions such as drug metabolism enzymes for 4 weeks in vitro. In some embodiments, the non-parenchymal cells include Kupffer cells. In some embodiments, the Kupffer cells are added to the hepatocyte-stromal cell bi-culture at day 7.

One drawback of existing hepatic co-culture technologies such as existing hepatocyte-Kupffer cell co-cultures is the inability of the individual cell populations to maintain functional stability during long-term culture. The inventors have found that allowing the hepatocytes to functionally stabilize in the micropatterned hepatocyte-stromal cell bi-culture prior to the addition of additional cell types such as but not limited to, Kupffer cells, allows the individual cell populations in the higher order cultures to survive and maintain optimal functional stability for a greater length of time. This is important to obtaining physiologically relevant interactions between the individual cell populations in the co-culture for better in vivo predictability and allows for a more accurate evaluation of various functions, such as, but not limited to, drug metabolism. In some embodiments, the Kupffer cells maintain functional stability in the tri-culture for at least 10 days. In some embodiments, the Kupffer cells maintain functional stability in the tri-culture for at least 4, 5, 6, 7, 8, 9, 10, 11, 12, 13 or 14 days. In some embodiments, the Kupffer cells maintain functional stability for at least 4-10, at least 4-11, at least 4-12, at least 4-13, at least 4-14, at least 5-10, at least 5-11, at least 5-12, at least 5-13, at least 5-14, at least 6-10, at least 6-11, at least 6-12, at least 6-13, at least 6-14, at least 7-10, at least 7-11, at least 7-12, at least 7-13 or at least 7-14 days. In some embodiments, the Kupffer cells maintain functional stability for about 4-10, about 4-11, about 4-12, about 4-13, about 4-14, about 5-10, about 5-11, about 5-12, about 5-13, about 5-14, about 6-10, about 6-11, about 6-12, about 6-13, about 6-14, about 7-10, about 7-11, about 7-12, about 7-13 or about 7-14 days. In some embodiments, the Kupffer cells maintain functional stability up to 10, 11, 12, 13, or 14 days. Functional stability of Kupffer cells may be determined by measuring Kupffer cell-specific functions such as, but not limited to, phagocytic activity, cytokine secretion, CYP450 inhibition, and cytotoxicity assays. Other means of evaluating Kupffer cell functionality include but are not limited to, immunostaining techniques such as CD68 staining (Brown et al. Am J Pathol. 15(6):2081-2088, 2001) and morphological assays. Various Kupffer-cell specific assays are known in the art and can be employed to evaluate the functional stability of Kupffer cells in culture. Advantages of the co-cultures disclosed herein include evaluating long-term effects of inflammation on drug metabolism and toxicity profiles.

In certain embodiments, hepatocytes and Kupffer cells maintain functional stability simultaneously. In some embodiments, the hepatocytes and Kupffer cells maintain functional stability in the tri-culture for at least 10 days. In some embodiments, the hepatocytes and Kupffer cells maintain functional stability for at least 4-10, at least 5-10, at least 6-10, or at least 7-14 days. In some embodiments, the hepatocytes and Kupffer cells maintain functional stability for about 4-10, about 5-10, about 6-10, or about 7-14 days. In some embodiments, the hepatocytes and the Kupffer cells maintain functional stability up to 10, 11, 12, 13, or 14 days.

Other non-parenchymal cells which may be added to the micropatterned hepatic co-cultures include, but are not limited to, liver cells such as Ito cells, sinusoidal endothelial cells, biliary duct cells, immune cells such as macrophages, T cells, neutrophils, dendritic cells, mast cells, eosinophils, and basophils, and stem cells such as liver progenitor cells, oval cells, hematopoietic stem cells, embryonic stem cells.

The stromal cells may be cells that have intrinsic attachment capabilities, thus eliminating a need for the addition of serum or exogenous attachment factors. Some cell types will attach to electrically charged cell culture substrates and will adhere to the substrate via cell surface proteins and by secretion of extracellular matrix molecules. The stromal cells may be fibroblasts (e.g., normal or transformed fibroblasts, such as NIH 3T3-J2 cells).

The cells can be primary cells, or they may be derived from an established cell line. The cell populations of the co-culture can be derived from one or more species. The cells may be mammalian cells, such as but not limited to human cells, rat cells, mouse cells, monkey cells, pig cells, dog cells, and guinea pig cells. In some embodiments, the cells are other vertebrate cells such as but not limited to, fish cells including zebrafish cells, or Xenopus cells. The cells may be fresh or cryopreserved.

In some embodiments, the micropatterned co-cultures are present in a multi-well format of up to 96-wells. In some embodiments, the micropatterned co-culture is located in a microfluidic device. In some embodiments, the micropatterned co-culture is located in tissue culture plate.

### (iv) IN VITRO MODEL OF LIVER INFLAMMATION

The micropatterned hepatic co-culture is capable of functioning as an in vitro model of liver inflammation. In one aspect, a micropatterned hepatic co-culture of hepatocytes and stromal cells is augmented with primary Kupffer macrophages to mimic one component of inflammation. The Kupffer cells may be added in multiple ratios. In some embodiments, the ratio of the Kupffer cells to the hepatocytes corresponds to the ratio of the cells in a physiologically normal state of the liver. In some embodiments, the ratio of the Kupffer cells to the hepatocytes corresponds to an inflamed state of the liver. It will be understood by one of skill in the art that such ratios are species and cell-type specific. For instance, human Kupffer cells may be added at the physiologic and inflammatory ratios of 0.1 and 0.4 to human hepatocytes, respectively. Rat Kupffer cells may be added at the physiologic and inflammatory ratios of 0.2 and 0.5 to rat hepatocytes, respectively. In some embodiments, human Kupffer cells are added at physiologic ratios of 0.075, 0.08, 0.085, 0.09, or 0.15. In some embodiments, human Kupffer cells are added at inflammatory ratios of 0.375, 0.38, 0.385, 0.39, 0.45, 0.475, 0.5 or 0.55. In some embodiments, rat Kupffer cells are added at physiologic ratios of 0.175, 0.18, 0.185, 0.19, 0.25. In some embodiments, rat Kupffer cells are added at inflammatory ratios of 0.475, 0.48, 0.485, 0.49, 0.55, 0.575, 0.6, or 0.65. Other non-parenchymal cells which may be added to the micropatterned hepatic co-culture inflammation model include, but are not limited to, liver cells such as Ito cells, sinusoidal endothelial cells, biliary duct cells, immune cells such as macrophages, T cells, neutrophils, dendritic cells, mast cells, eosinophils, and basophils, and stem cells such as liver progenitor cells, oval cells, hematopoietic stem cells, embryonic stem cells.

The co-culture platform of the invention can be used with a number of different cytokines and other inflammation-inducing agents to generate the desired inflammation model. In some embodiments, the inflammation-inducing agent is bacterial lipopolysaccharide or endotoxin (LPS). LPS is known to induce Kupffer cells to secrete inflammatory cytokines. In some embodiments, the inflammation-inducing agent is a cytokine such as, but not limited to, TNF-α, TNF-β, IL-1, IL-6, IL-8, IL-12, IL-15, IL-18, MIP-1α, MIP-1β, MCP-1, IFNγ, IL-2, IFNα/β, lymphotoxinαβ, LIGHT, CD40L, FasL, CD30L, CD27L, 4-1BBL, Ox40L, CD120α, and CD120β. In some embodiments, the inflammation-inducing agent is a cytokine, such as, but not limited to, IL-1B. The co-culture platform can be easily adapted for use with various inflammation-inducing agents.

The in vitro liver inflammation model of the invention has significant in vivo and in vitro investigative potential and provides a unique insight into the role of the immune system in drug-drug interactions. The in vitro liver inflammation model of the invention has utility in detecting and evaluating inflammation-drug interactions and inflammation-mediated toxicities. In some cases, the compounds are known to cause immune-mediated liver toxicities. In some embodiments, the compounds are tested in a pre-clinical setting. In one aspect, the in vitro liver inflammation model of the invention has utility in evaluating the mechanisms underlying inflammation-mediating toxicities. The in vitro liver inflammation model of the invention also has utility in assessment of clinically relevant interactions between compounds such as, but not limited to, therapeutic biologics and small molecule drugs. In some embodiments, functional stability of the in vitro liver inflammation model of the invention allows for evaluating long-term effects of inflammation on test compounds and is predictive of in vivo results.

In one aspect, in vitro liver inflammation model of the invention provides a platform to evaluate effects of inflammation on the metabolism of co-administered drugs, when one of the drugs results in the appearance or relief of inflammation. Other sources of inflammation include, but are not limited to, pathogenic infections and autoimmune disorders.

The micropatterned co-cultures of the invention are useful in drug discovery and development including screening for metabolic stability, drug-drug interactions, and toxicity. Metabolic stability is a key criterion for selection of lead drug candidates that proceed to preclinical trials. The in vitro liver inflammation model of the invention is useful in understanding how inflammation alters drug metabolism, toxicity and drug-drug interactions. In one aspect, the in vitro liver inflammation model of the invention is useful for long-term evaluation of the effects of inflammation-mediated drug toxicity.

Due to species-specific differences in drug metabolism, human hepatocyte cultures can identify the metabolite profiles of drug candidates more effectively than non-human cultures. Although, it will be recognized that non-human cell types may be used in the invention to facilitate identification of properties or metabolisms suitable for further study of human cells. Non-human cell types include but are not limited to rat, mouse, monkey, pig, dog, guinea pig, fish, and Xenopus. This information can then be used to deduce the mechanism by which the metabolites are generated, with the ultimate goal of focusing clinical studies. Though there are quantitative differences, there is good in vivo to in vitro correlation in drug biotransformation activity when isolated hepatocytes are used. Metabolite profiles obtained via human hepatocyte in vitro models can also be used to choose the appropriate animal species to act as the human surrogate for preclinical pharmacokinetic, pharmacodynamic and toxicological studies. Studies have shown that interspecies variations are retained in vitro and are different depending on the drug being tested.

The validation of human co-cultures as appropriate liver models for drug development includes cell-based acute and chronic toxicity assays using a variety of clinical and non-clinical compounds, as well as induction and inhibition of key CYP450 enzymes.

The disclosure provides methods of determining the inflammation-mediated effects on compound-hepatocyte interactions, including but not limited to, determining inflammation-mediated effects on metabolic stability, drug-drug interactions, and toxicity.

The co-cultures of the disclosure may be used to in vitro to screen a wide variety of compounds, such as cytotoxic compounds, growth/regulatory factors, pharmaceutical agents, and the like, to identify agents that modify cell (e.g., hepatocyte) function and/or cause cytotoxicity and death or modify proliferative activity or cell function. In some embodiments, the co-cultures of the disclosure may be used to screen a wide variety of compounds, such as cytotoxic compounds, growth/regulatory factors, pharmaceutical agents, and the like, to identify agents that modify cell (e.g., hepatocyte) function and/or cause cytotoxicity and death or modify proliferative activity or cell function whose metabolic, toxicity and/or drug-drug interaction profiles are significantly altered by inflammation. In some embodiments, the co-cultures are used to identify compounds that have the potential to exhibit idiosyncratic liver toxicity. For example, the culture system may be used to test adsorption, distribution, metabolism, excretion, and toxicology (ADMET) of various agents in the presence or absence of inflammation. To this end, the cultures are maintained in vitro comprising a defined cellular island geometry and exposed to a compound to be tested. The activity of a compound can be measured by its ability to damage or kill cells in culture or by its ability to modify the function of the cells (e.g., in hepatocytes the expression of P450, and the like). This may readily be assessed by vital staining techniques, ELISA assays, immunohistochemistry, and the like. The effect of growth/regulatory factors on the cells (e.g., hepatocytes) may be assessed by analyzing the cellular content of the culture, e.g., by total cell counts, and differential cell counts or by metabolic markers such as MTT and XTT. This may also be accomplished using standard cytological and/or histological techniques including the use of immunocytochemical techniques employing antibodies that define type-specific cellular antigens.

In one aspect, the activity of a compound can be measured by its effect on gene expression, cell function, metabolic activity, morphology, or a combination thereof, of the hepatocytes of the co-culture. In some embodiments, the metabolic activity is Phase I or Phase II enzyme activity, urea synthesis, or albumin secretion. In some embodiments, the effect on CYP450 expression or activity is measured. In some embodiments, the CYP450 isoenzyme is CYP3A4. Exemplary CYP450 isoenzymes include CYP1A2, CYP1B1, CYP2A6, CYP2B6, CYP2D6, CYP2E1, CYP2F1, CYP2J2, CYP3A4, CYP4A, and CYP4B. Exemplary CYP450 isoenzymes are described in U.S. Pat. No. 8,217,161, the disclosure of which is incorporated herein by reference in its entirety. In some embodiments, the effect on hepatic uptake is measured. In some embodiments, effect on cell function is assessed by measuring ATP levels. In some embodiments, cytokine secretion is measured. Cytokine arrays may be used to measure cytokine release. In some embodiments protein secretion is analyzed. To detect the modulation of a metabolic or synthetic function, conventional molecular and biochemical assays can be used. For example, suitable in vitro assays can include assays to analyze hepatocyte proliferation, e.g., via BrdU incorporation; hepatocyte apoptosis, for example, by analyzing morphological changes associated with apoptosis/necrosis, or by using, e.g., TUNEL assay; RT-PCR to detect alterations in the mRNA expression levels of IL-1β, IL-6, HGF, EGF, and TNF-α, ELISA to detect altered IL-1β, TNF-α, IL-1β, IL-6, IL-2, IL-1ra expression.

In one aspect, characteristics of a compound are measured in the presence or absence of inflammation. In some embodiments, metabolic stability of a compound is measured. In some embodiments, presence or absence of metabolites is measured. In some embodiments, the compound may be detectably labeled, for example, the compound may be radiolabeled. Methods for measuring characteristics of compounds, such as mass spectrometry, are well known in the art.

The cytotoxicity to cells in culture (e.g., human hepatocytes) of pharmaceuticals, anti-neoplastic agents, carcinogens, food additives, and other substances may be tested by utilizing the co-culture system of the disclosure in the presence or absence of inflammation. In certain embodiments, toxicity may be mediated by TNF-α or IL-6. In certain embodiments, toxicity may be mediated by TNF-α, TNF-β, IL-1, IL-6, IL-8, IL-12, IL-15, IL-18, MIP-1α, MIP-1β, MCP-1, IFNγ, IL-2, IFNα/β, lymphotoxinαβ, LIGHT, CD40L, FasL, CD30L, CD27L, 4-1BBL, Ox40L, CD120α, or CD120β.

In one aspect, cytotoxicity is measured by generating a dose-response curve to determine the 50% toxic concentration (TC50) value. The length of dosing and the range of dosing concentrations will vary depending on the compound. In one embodiment, the cultures are dosed with the compounds at multiples of the maximum plasma concentration (Cmax) of the compound. In one embodiment, cytoxicity is determined by measuring cellular ATP content. In some embodiments, cytotoxicity is determined by total cell counts, and differential cell counts or by metabolic markers such as MTT and XTT, Resazurin conversion, or alamarBlue assay (Life Technologies). In one aspect, cytotoxicity in the presence or absence of inflammation is determined to determine if cytotoxicity is potentiated by inflammation. In one embodiment, cytotoxicity may be potentiated by inflammation if the TC50 value in the presence of inflammation is lowered by at least 20%, at least 30%, at least 40%, at least 50%, at least 60%, at least 70%, at least 80%, or at least 90%. In some embodiments, the TC50 value in the presence of inflammation is lowered by 20-35%, 35-50%, 50-65%, 65-80%, or 80-90%.

In one aspect of the assay system, a stable, growing culture is established having a desired size (e.g., island size and distance between islands), morphology and may also include a desired oxygen gradient. The cells/tissue in the culture are exposed to varying concentrations of a test agent. After incubation with a test agent, the culture is examined by phase microscopy or by measuring cell specific functions (e.g., hepatocyte cell indicators) such as protein production/metabolism to determine the highest tolerated dose—the concentration of test agent at which the earliest morphological abnormalities appear or are detected. Cytotoxicity testing can be performed using a variety of supravital dyes to assess cell viability in the culture system, using techniques known to those skilled in the art.

Once a testing range is established, varying concentrations of the test agent can be examined for their effect on viability, growth, and/or morphology of the different cell types.

In one aspect, the micropatterned co-cultures of the invention can be scaled-up to form a high-throughput microreactor array to allow for interrogation of xenobiotics. In one aspect, a microfluidic device is contemplated that has micropatterned co-culture areas in or along a fluid flow path.

Similarly, the beneficial effects of drugs may be assessed using the culture system in vitro; for example, growth factors, hormones, drugs which enhance hepatocyte formation or activity can be tested. In this case, stable micropattern cultures may be exposed to a test agent. After incubation, the micropattern cultures may be examined for viability, growth, morphology, cell typing, and the like as an indication of the efficacy of the test substance. Varying concentrations of the drug may be tested to derive a dose-response curve.

The culture systems of the invention may be used as model systems for the study of physiologic or pathologic conditions. For example, in a specific embodiment, the culture system can be optimized to act in a specific functional manner as described herein by modifying the size or distribution of cellular islands. In another aspect, the oxygen gradient is modified along with the density and or size of a micropattern of cells in the culture system.

One advantage of the culture systems of the disclosures is that the cells in such a culture system are substantially homogenous and autologous (e.g., the cellular islands are substantially homogenous and autologous) so you can do many experiments on the same biological background. In vivo testing, for example, suffers from animal-to-animal variability and is limited by the number of conditions or agents that can be tested on a given subject.

The compounds to be tested in the methods of the disclosure include, but are not limited to, pharmaceutical agents, pharmaceuticals, anti-neoplastic agents, carcinogens, food additives, xenobiotics, and cytotoxic agents. In some embodiments, the test compound is a small molecule, protein, protein fragment, or peptide, In some embodiments, the test compound is a small molecule of MW 1000-2000, MW 2000-2500, MW 2500-3000.

To demonstrate applications in drug development, acute acute/chronic toxicity assays as well as induction/inhibition of cytochrome P450s (key drug metabolism enzymes) via Trovafloxacin, which causes an inflammation-mediated idiosyncratic liver toxicity were tested. Results show that this drug toxicity in the inflammation model is potentiated with LPS. Accordingly, the invention is useful to screen for toxicity and drug interactions the may have either positive or negative effects on cellular metabolism.

### (v) METHODS OF MICROPATTERNING

Methods of micropatterning useful to develop co-cultures with desired characteristics are described in U.S. Pat. No. 6,133,030 and U.S. Patent Application No. 2006-0270032, the disclosures of which are incorporated herein by reference in their entirety.

The cellular islands can take any geometric shape having a desired characteristic and can be defined by length/width, diameter and the like, based upon their geometric shape, which may be circular, oval, square, rectangular, triangular and the like. Furthermore, parenchymal cell (e.g. hepatocyte) function may be modified by altering the pattern configuration (e.g., the distance or geometry of the array of cellular islands). The distance between bounded geometric islands of cells may vary in a culture system (e.g., the distances between islands may be regular or irregular). Using techniques described herein, the spatial distances between cellular islands may be random, regular or irregular. Furthermore, combinations of geometric bounded areas (e.g., cellular islands) of different geometries (e.g., multiple island sizes) may be present on a single substrate with varying distances (e.g., multiple island spacings) or regular distances between the islands. In other words, the invention contemplates the use of cellular islands comprising various geometries and distances on a substrate (e.g., cocultures comprising cellular islands with 250 μm and 400 μm islands that are intermixed and regularly distributed). In one aspect, the cellular islands comprise a diameter or width from about 250 μm to 750 μm. Similarly, where the geometric island comprises a rectangle, the width can comprise about 250 μm to 750 μm. In another aspect, the parenchymal cellular islands are spaced apart from one another by about 2 μm to 1300 μm from center to center of the cellular islands. In yet a further aspect, the parenchymal cell islands comprise a defined width (e.g., 250 μm to 750 μm) that can run the length of a culture area or a portion of the culture area. Parallel islands of parenchymal cells can be separated by parallel rows of stromal cells. In another aspect, the geometric shape may comprise a 3-D shape (e.g., a spheroid). In such instances, the diameter/width and the like, will be from about 250 μm to 750 μm. Additional non-parenchymal cells can be seeded at multiple ratios to allow balance of homotypic (hepatocyte/hepatocyte) and heterotypic (hepatocyte/stroma or hepatocyte/Kupffer cell) interactions in the micropatterned co-culture.

As will be recognized in the art, the cellular islands may be present in any culture system including static and fluid flow reactor systems (e.g., microfluidic devices). Such microfluidic devices are useful in the rapid screening of agents where small flow rates and small reagent amounts are required.

The cellular culture of the invention can be made by any number of techniques that will be recognized in the art. For example, a method of making a plurality of cellular islands on a substrate can comprise spotting or layering an adherence material (or plurality of different cell specific adherence materials) on a substrate at spatially different locations each spot having a defined size (e.g., diameter) and spatial arrangement. The spots on the substrate are then contacted with a first cell population or a combination of cell types and cultured to generate cellular islands. Where difference cell-types are simultaneously contacted with the substrate, the substrate, coating or spots on the substrate will support cell-specific binding, thus providing distinct cellular domains. Methods for spotting adherence material (e.g., extracellular matrix material) can include, for example, robotic spotting techniques and lithographic techniques.

Various culture substrates can be used in the methods and systems of the invention. Such substrates include, but are not limited to, glass, polystyrene, polypropylene, stainless steel, silicon and the like. The choice of the substrate should be taken into account where spatially separated cellular islands are to be maintained. The cell culture surface can be chosen from any number of rigid or elastic supports. For example, cell culture material can comprise glass or polymer microscope slides. In some aspect, the substrate may be selected based upon a cell type's propensity to bind to the substrate.

The cell culture surface/substrate used in the methods and systems of the invention can be made of any material suitable for culturing mammalian cells. For example, the substrate can be a material that can be easily sterilized such as plastic or other artificial polymer material, so long as the material is biocompatible. A substrate can be any material that allows cells and/or tissue to adhere (or can be modified to allow cells and/or tissue to adhere or not adhere at select locations) and that allows cells and/or tissue to grow in one or more layers. Any number of materials can be used to form the substrate/surface, including, but not limited to, polyamides; polyesters; polystyrene; polypropylene; polyacrylates; polyvinyl compounds (e.g. polyvinylchloride); polycarbonate (PVC); polytetrafluoroethylene (PTFE); nitrocellulose; cotton; polyglycolic acid (PGA); cellulose; dextran; gelatin, glass, fluoropolymers, fluorinated ethylene propylene, polyvinylidene, polydimethylsiloxane, polystyrene, and silicon substrates (such as fused silica, polysilicon, or single silicon crystals), and the like. Also metals (gold, silver, titanium films) can be used.

As mentioned herein, in some instances the substrate may be modified to promote cellular adhesion and growth (e.g., coated with an adherence material). For example, a glass substrate may be treated with a protein (i.e., a peptide of at least two amino acids) such as collagen or fibronectin to assist cells in adhering to the substrate. In some embodiments, the proteinaceous material is used to define the location of a cellular island. The spot produced by the protein serves as a “template” for formation of the cellular island. Typically, a single protein will be adhered to the substrate, although two or more proteins may be used in certain embodiments. Proteins that are suitable for use in modifying a substrate to facilitate cell adhesion include proteins to which specific cell types adhere under cell culture conditions. For example, hepatocytes are known to bind to collagen. Therefore, collagen is well suited to facilitate binding of hepatocytes. Other suitable proteins include fibronectin, gelatin, collagen type IV, laminin, entactin, and other basement proteins, including glycosaminoglycans such as heparin sulfate. Combinations of such proteins also can be used.

The type of adherence material(s) (e.g., ECM materials, sugars, proteoglycans etc.) deposited in a spot will be determined, in part, by the cell type or types to be cultured. For example, ECM molecules found in the hepatic microenvironment are useful in culturing hepatocytes, the use of primary cells, and a fetal liver-specific reporter ES cell line. The liver has heterogeneous staining for collagen I, collagen III, collagen IV, laminin, and fibronectin. Hepatocytes display integrins β1, β2, α1, α2, α5, and the nonintegrin fibronectin receptor Agp110 in vivo. Cultured rat hepatocytes display integrins α1, α3, α5, α1, and α6 μ1, and their expression is modulated by the culture conditions.

The total number of spots on the substrate will vary depending on the substrate size, the size of a desired cellular island, and the spacing between cellular islands. Generally, the pattern present on the surface of the support will comprise at least 2 distinct spots, usually about 10 distinct spots, and more usually about 100 distinct spots, where the number of spots can be as high as 50,000 or higher. Typically, the spot will usually have an overall circular dimension (although other geometries such as spheroids, rectangles, squares and the like may be used) and the diameter will range from about 10 to 5000 μm (e.g., about 200 to 800 μm). The cellular islands may be spaced apart 1200 μm to 1300 μm from center to center of the cellular islands. In some embodiments, the cellular islands may be spaced apart 1100-1200 μm, 1200-1300 μm, 1300-1350 μm, 1350-1450 μm, or 1450-1500 μm.

By dispensing or printing onto the surfaces of multi-well culture plates, one can combine the advantages of the array approach with those of the multi-well approach. Typically, the separation between tips in standard spotting device is compatible with 96 well plates; one can simultaneously print each load in several wells. Printing into wells can be done using both contact and non-contact technology. The cell density is typically around 5000 per well. In some embodiments, the cell density may be 3500-5000, 5000-5500, 5500-6000, 6000-7500.

The invention can utilize robotic spotting technology to develop a robust, accessible method for forming cellular microarrays or islands of a defined size and spatial configuration on, for example, a cell culture substrate. As used herein, the term “microarray” refers to a plurality of addressed or addressable locations.

In one aspect, the invention provides methods and systems comprising a modified printing buffer used in a spotting device to allow for ECM deposition, and identifying microarray substrates that permit ECM immobilization. The methods and systems of the invention are useful for spotting substantially purified or mixtures of biological proteins, nucleic acids and the like (e.g., collagen I, collagen III, collagen IV, laminin, and fibronectin) in various combinations on a standard cell culture substrate (e.g., a microscope slide) using off-the-shelf chemicals and a conventional DNA robotic spotter.

In another aspect, the invention utilizes photolithographic techniques to generate cellular islands. Drawing on photolithographic micropatterning techniques to manipulate functions of rodent hepatocytes upon co-cultivation with stromal cells, a microtechnology-based process utilizing elastomeric stencils to miniaturize and characterize human liver tissue in an industry-standard multiwell format was used. The approach incorporates ‘soft lithography,’ a set of techniques utilizing reusable, elastomeric, polymer (Polydimethylsiloxane-PDMS) molds of microfabricated structures to overcome limitations of photolithography. In one aspect, the invention provides a process using PDMS stencils consisting of 300 μm thick membranes with through-holes at the bottom of each well in a 24-well mold. To micropattern all wells simultaneously, the assembly was sealed against a polystyrene plate. Collagen-I was physisorbed to exposed polystyrene, the stencil was removed, and a 24-well PDMS ‘blank’ was applied. Co-cultures were ‘micropatterned’ by selective adhesion of human hepatocytes to collagenous domains, which were then surrounded by supportive murine 3T3-J2 fibroblasts. The size (e.g., geometric dimension) of through-holes determined the size of collagenous domains and thereby the balance of homotypic (hepatocyte/hepatocyte) and heterotypic (hepatocyte/stroma or hepatocyte/Kupffer cell) interactions in the microscale tissue. Similar techniques can be used to culture cellular islands of other parenchymal cell types.

The invention provides methods and systems useful for identifying optimal conditions for controlling cellular development and maturation by varying the size and/or spacing of a cellular island. For example, the methods and systems of the invention are useful for identifying optimal conditions that control the fate of cells (e.g., differentiating stem cells into more mature cells, maintenance of self-renewal, and the like).

The term “adherence material” is a material deposited on a substrate or chip to which a cell or microorganism has some affinity, such as a binding agent. The material can be deposited in a domain or “spot”. The material and a cell or microorganism interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, sugars, proteoglycans, or cellular receptors.

### (vi) CELLULAR COMPONENTS OF THE CO-CULTURE

Cells useful in the methods of the disclosure are available from a number of sources including commercial sources. For example, hepatocytes may be isolated by conventional methods (Berry and Friend, 1969, J. Cell Biol. 43:506-520) which can be adapted for human liver biopsy or autopsy material. Typically, a cannula is introduced into the portal vein or a portal branch and the liver is perfused with calcium-free or magnesium-free buffer until the tissue appears pale. The organ is then perfused with a proteolytic enzyme such as a collagenase solution at an adequate flow rate. This should digest the connective tissue framework. The liver is then washed in buffer and the cells are dispersed. The cell suspension may be filtered through a 70 μm nylon mesh to remove debris. Hepatocytes may be selected from the cell suspension by two or three differential centrifugations.

For perfusion of individual lobes of excised human liver, HEPES buffer may be used. Perfusion of collagenase in HEPES buffer may be accomplished at the rate of about 30 ml/minute. A single cell suspension is obtained by further incubation with collagenase for 15-20 minutes at 37° C. (Guguen-Guillouzo and Guillouzo, eds, 1986, “Isolated and Culture Hepatocytes” Paris, INSERM, and London, John Libbey Eurotext, pp. 1-12; 1982, Cell Biol. Int. Rep. 6:625-628).

Hepatocytes may also be obtained by differentiating pluripotent stem cell or liver precursor cells (i.e., hepatocyte precursor cells). The isolated hepatocytes may then be used in the culture systems described herein.

Cryopreserved human hepatocytes and fresh human Kupffer cells can be obtained from Celsis In Vitro Technologies. Fresh human Kupffer cells can be cryopreserved by Hepregen Corporation. Cryopreserved rat Kupffer cells can be obtained from Life Technologies.

Stromal cells include, for example, fibroblasts obtained from appropriate sources as described further herein. Alternatively, the stromal cells may be obtained from commercial sources or derived from pluripotent stem cells using methods known in the art.

Fibroblasts may be readily isolated by disaggregating an appropriate organ or tissue which is to serve as the source of the fibroblasts. This may be readily accomplished using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include, but are not limited to, trypsin, chymotrypsin, collagenase, elastase, and/or hyaluronidase, DNase, pronase, dispase and the like. Mechanical disruption can also be accomplished by a number of methods including, but not limited to, the use of grinders, blenders, sieves, homogenizers, pressure cells, or insonators. For a review of tissue disaggregation techniques, see Freshney, Culture of Animal Cells. A Manual of Basic Technique, 2d Ed., A. R. Liss, Inc., New York, 1987, Ch. 9, pp. 107-126.

Once the tissue has been reduced to a suspension of individual cells, the suspension can be fractionated into subpopulations from which the fibroblasts and/or other stromal cells and/or elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis, fluorescence-activated cell sorting, and the like. For a review of clonal selection and cell separation techniques, see Freshney, Culture of Animal Cells. A Manual of Basic Techniques, 2d Ed., A. R. Liss, Inc., New York, 1987, Ch. 11 and 12, pp. 137-168.

The isolation of fibroblasts can, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. All fibroblasts will attach before other cells, therefore, appropriate stromal cells can be selectively isolated and grown. The isolated fibroblasts can then be used in the culture systems of the disclosure.

Cancer tissue may also be cultured using the methods and co-culture system of the disclosure. For example, adenocarcinoma cells can be obtained by separating the adenocarcinoma cells from stromal cells by mincing tumor cells in HBSS, incubating the cells in 0.27% trypsin for 24 hours at 37° C. and further incubating suspended cells in DMEM complete medium on a plastic petri dish for 12 hours at 37° C. Stromal cells selectively adhered to the plastic dishes.

The co-cultures of the disclosure may be used to study cell and tissue morphology. For example, enzymatic and/or metabolic activity may be monitored in the culture system remotely by fluorescence or spectroscopic measurements on a conventional microscope.

In one aspect, a fluorescent metabolite in the fluid/media is used such that cells will fluoresce under appropriate conditions (e.g., upon production of certain enzymes that act upon the metabolite, and the like). Alternatively, recombinant cells can be used in the cultures system, whereby such cells have been genetically modified to include a promoter or polypeptide that produces a therapeutic or diagnostic product under appropriate conditions (e.g., upon zonation or under a particular oxygen concentration). For example, a hepatocyte may be engineered to comprise a GFP (green fluorescent protein) reporter on a P450 gene (CYPIA1). Thus, if a drug activates the promoter, the recombinant cell fluoresces. This is useful for predicting drug-drug interactions that occur due to upregulation in P450s.

The various techniques, methods, and aspects of the invention described above can be implemented in part or in whole using computer-based systems and methods. For example, computer implemented methods can be used in lithography techniques to design cellular islands.

The disclosure provides co-cultures of cells in which at least two types of cells are configured in a bounded geometric pattern on a substrate. Such micropatterning techniques are useful to modulate the extent of heterotypic and homotypic cell-cell contacts. In addition, co-cultures have improved stability and thereby allow chronic testing (e.g., chronic toxicity testing as required by the Food and Drug Administration for new compounds). Because micropatterned co-cultures are more stable than random cultures the use of co-cultures of the invention and more particularly micropatterned co-cultures provide a beneficial aspect to the cultures systems of the disclosure. Furthermore, because drug-drug interactions often occur over long periods of time the benefit of stable co-cultures allows for analysis of such interactions and toxicology measurements.

In one aspect, the invention provides an in vitro model of human liver tissue that can be utilized for pharmaceutical drug development, basic science research, infectious disease research (e.g., hepatits B, C and malaria) and in the development of tissue for transplantation. The invention provides compositions, methods, and co-culture systems that allow development of long-term human cultures in vitro. In addition, the compositions, methods and co-culture systems of the invention provide for the design of particular morphological characteristics by modifying cellular island size and distribution, and individual cell population ratios. The compositions, methods and co-culture systems of the disclosure have been applied to liver cultures and have shown that cellular island size and/or distribution contribute to induction of cellular metabolism that mimics in vivo metabolism. The results demonstrate that cellular distribution modulates gene expression and imply an important role in the maintenance of cell-specific metabolism (e.g., liver specific metabolism). In addition, considerations of the effect of such distribution in the design and optimization of current bioartificial support systems may serve to improve their function.

Certain materials, such as nylon, polystyrene, and the like, are less effective as substrates for cellular and/or tissue attachment. When these materials are used as the substrate it is advisable to pre-treat the substrate prior to inoculation with cells in order to enhance the attachment of cells to the substrate. For example, prior to inoculation with stromal cells and/or parenchymal cells, nylon substrates should be treated with 0.1 M acetic acid, and incubated in polylysine, FBS, and/or collagen to coat the nylon. Polystyrene could be similarly treated using sulfuric acid.

### EXEMPLIFICATION

The working examples provided below are to illustrate, not limit, the disclosure. Various parameters of the scientific methods employed in these examples are described in detail below and provide guidance for practicing the disclosure in general.

In these particular working examples, hepatocytes are co-cultured with fibroblasts and Kupffer cells. Similar methods can be used to co-culture other combinations of cells. Although the invention has been generally described above, further aspects of the invention will be apparent from the specific disclosure that follows, which is exemplary and not limiting.

### Example 1

### Hepatocyte-Stromal Cell-Kupffer Cell Co-Cultures

Cryopreserved human hepatocytes, cryopreserved human Kupffer cells and 3T3 J2 mouse fibroblasts were used in the manufacturing of the co-culture platform. Cryopreserved hepatocyte vials were thawed in a 37° C. water bath for 90-120 seconds followed by dilution in 50 mL of pre-warmed percoll-based hepatocyte density sedimentation medium (p=1.06). The cell suspension was spun at 50×g for 5 minutes. The supernatant was discarded, cells were re-suspended in hepatocyte culture media (HCM) (Hepregen Corporation, Medford, Mass.) and viability was assessed using trypan blue exclusion (typically 80-95%). Liver-derived non-parenchymal cells, as judged by their size (˜10 μm in diameter) and morphology (non-polygonal), were consistently found to be less than 1% in these preparations. To create micropatterned co-cultures (HepatoPac™, Hepregen Corporation) in 96-well plates, a hepatocyte pattern was first produced by seeding hepatocytes on rat-tail collagen (BD Biosciences, Franklin Lakes, N.J.) type I-patterned substrates that mediate selective cell adhesion. The cells were washed with medium 4-6 hours later to remove unattached cells (leaving 5,000 attached hepatocytes on 13 collagen-coated islands within each well of a 96-well plate) and incubated in HCM. 3T3-J2 murine embryonic fibroblasts were seeded 12-18 h later to create co-cultures. Culture medium was replaced every 2 days (−65 μL per well) for 7 days. Cryopreserved human Kupffer cells from unmatched donor were thawed and seeded (on Day 7 of HepatoPac™ culture) at a ratio of 1:0.4 (hepatocytes: Kupffer cells). Kupffer cells were partially thawed in a 37 degree Celsius water bath until a sliver of ice was visible in each vial (per manufacturer's specifications). Kupffer cells were then resuspended in 15 ml of cold HCM and spun at 800×g. Supernatant above the Kupffer cell pellet was removed and enough cold HCM was added to bring expected Kupffer cell yield to 1 million cell/ml. Kupffer cells were gently rocked into suspension and evaluated for viability via Trypan Blue exclusion. Media was removed from the HepatoPac™ cultures and Kupffer cells were seeded chilled on top of the HepatoPac™ cultures at the desired ratio (1:0.1 or 1:0.4) at a volume of 50 ul per 96-well. Culture plates were placed in the incubator and gently shaken every 30 minutes for 2 hours and remained in the incubator undisturbed overnight. All reagents used in the study were of analytical grade.

Cytotoxicity was assessed by evaluation of cellular ATP content while hepatocyte metabolic competence was assessed by urea production. Production of urea, a liver-specific function, was assayed in culture media using the Stanbio kit according to manufacturer's directions. The reaction is based on the acid catalyzed condensation of urea with diacetylmonoxime in the presence of thiosemicarbazide to yield a red purple chromogen that is read spectrophotometrically. The cellular ATP content was measured in cell lysates using the Cell Titer-Glo luminescent kit from Promega (Madison, Wis.).

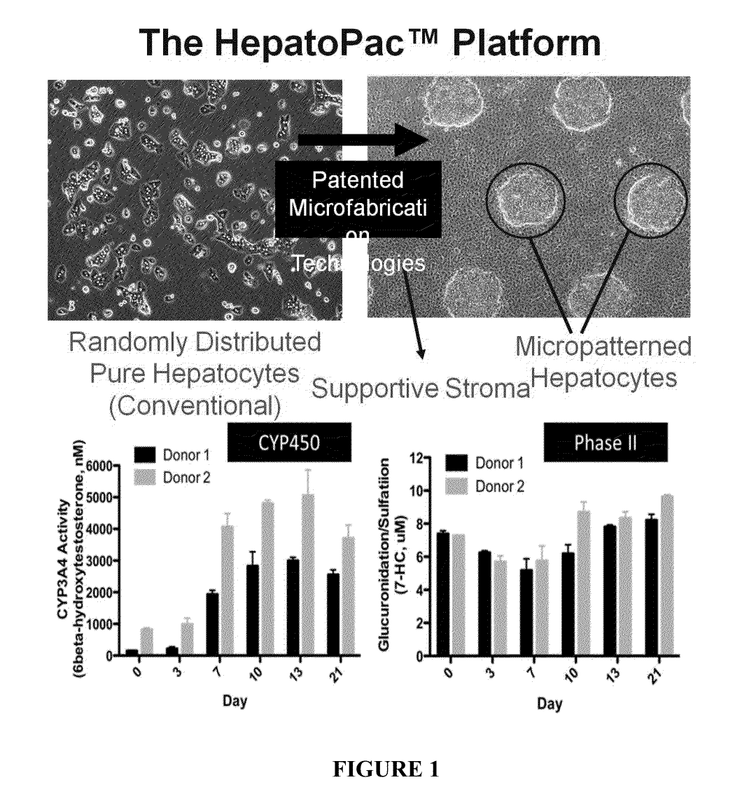

The HepatoPac™ platform is shown in FIG. 1. Human HepatoPac™ cultures created using cryopreserved human hepatocyte donors retain long-term functionality (i.e. Phase I and II drug metabolism enzymes) for several weeks in vitro (FIG. 1). Here, CYP3A4 activity via testosterone 6β-hydroxylation is shown. Phase II metabolism is shown here via glucuronidation and sulfation of 7-hydroxycoumarin.

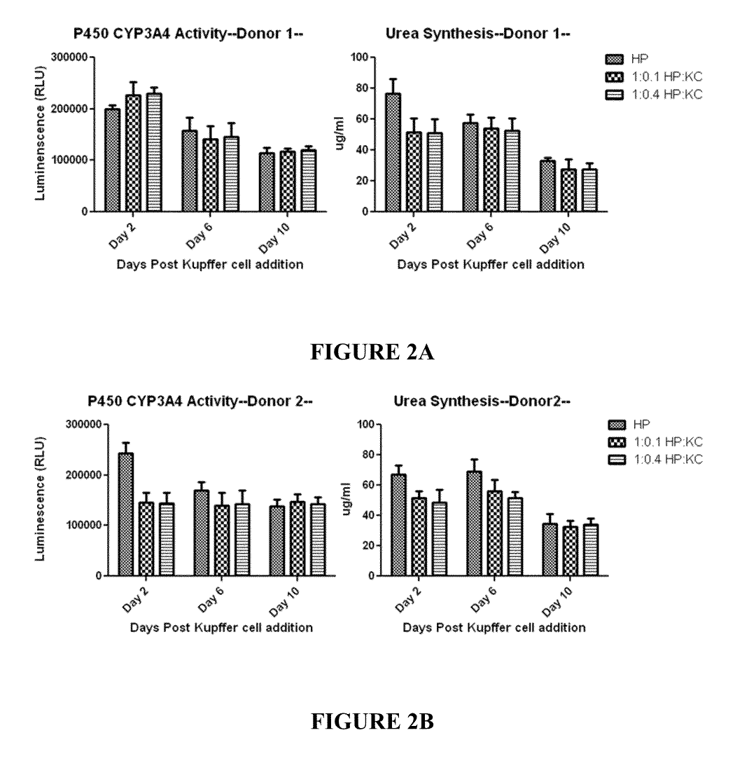

The HepatoPac™ platform is augmented with primary kupffer macrophages in order to mimic one component of inflammation. Kupffer cells were added to human HepatoPac™ at multiple ratios (to mimic both the normal and inflamed state of the liver) after stabilization to generate a tri-culture with human hepatocytes and murine embryonic fibroblasts (HepatoPac™-kupffer co-culture). Assessment of hepatocyte metabolic competence in the presence or absence of Kupffer cells showed comparable production of albumin (not shown) and urea suggesting the presence of functional hepatocytes in co-cultures. FIGS. 2A and 2B show that addition of Kupffer cells to HepatoPac™ does not have a significant effect on hepatocyte functionality as determined here by CYP3A4 activity or Urea Synthesis measured 2, 6 and 10 days after.

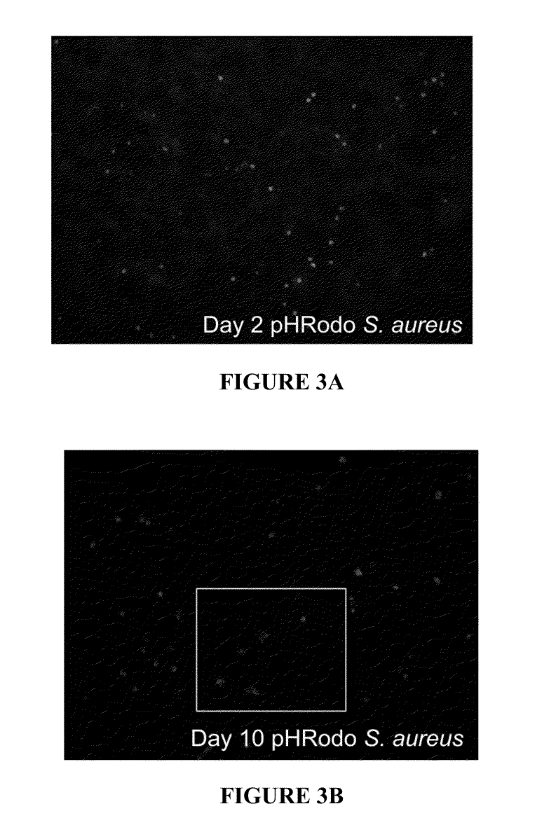

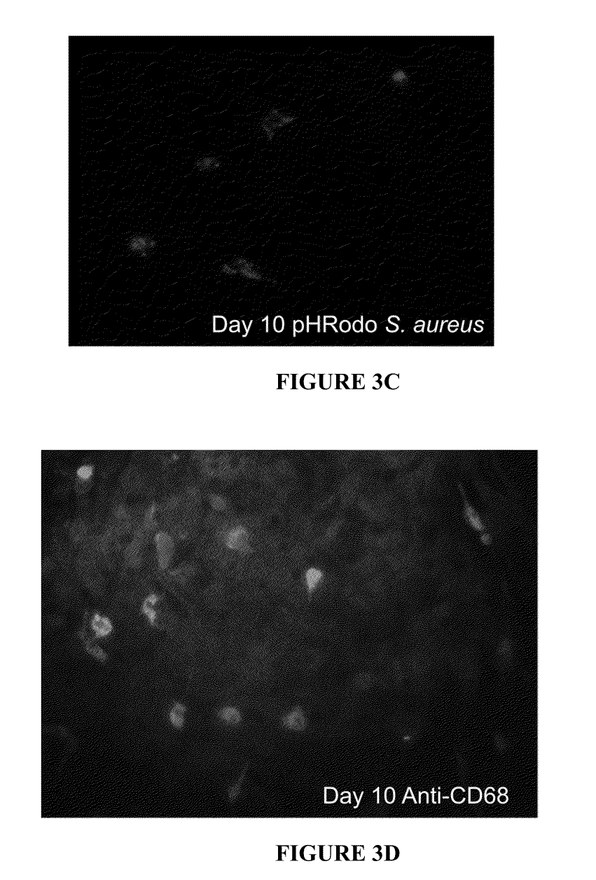

In addition, Kupffer cell functionality is maintained in the co-culture. FIGS. 3A-3D show validation of Kupffer cell functionality in co-culture. As shown in red in FIGS. 3A-3C, Kupffer cells in co-culture are specifically able to phagocytose pHRODO Red labeled S. aureus bioparticles. Phagocytosis, which is an important marker for functional Kupffer cells, is detected at 2 days and as late as 10 days post Kupffer cell addition (17 days of HepatoPac™ culture). FIG. 3D shows immuno-staining with anti-CD68 in green, which confirms the presence of Kupffer cells in co-culture as late as 10 days after the addition of Kupffer cells. Phagocytosis has been observed as late as 14 days after the addition of Kupffer cells (not shown). Kupffer cells in the tri-culture, therefore, remain viable for upwards of 10 days, exhibiting both CD68 surface markers and positive phagocytosis of pH-sensitive S. aureus bioparticles. Macrophage responsiveness to endotoxin (LPS) and the inflammatory signaling molecule IL-1B, as measured by IL-6 secretion, as shown below, persisted for the 5 days investigated in this study. Methods of IL-6 mediated CYP3A4 down-regulation in hepatocytes through LPS and IL-1B-induced secretion of IL-6 by the Kupffer cells are presented below.

### Example 2

### Responsiveness to LPS Stimulation

Fibroblast and hepatocyte co-cultures were stabilized in serum-positive media for 7 days prior to Kupffer cell seeding at the physiologic and inflammatory ratios of 0.1 and 0.4 Kupffer cells to hepatocytes, respectively. Lipopolysaccharide (LPS) stimulation of cultures occurred overnight for 20 hours at 50 ng/ml after which supernatants were analyzed for cytokine secretion using BD OptEIA ELISA kits (Sigma).

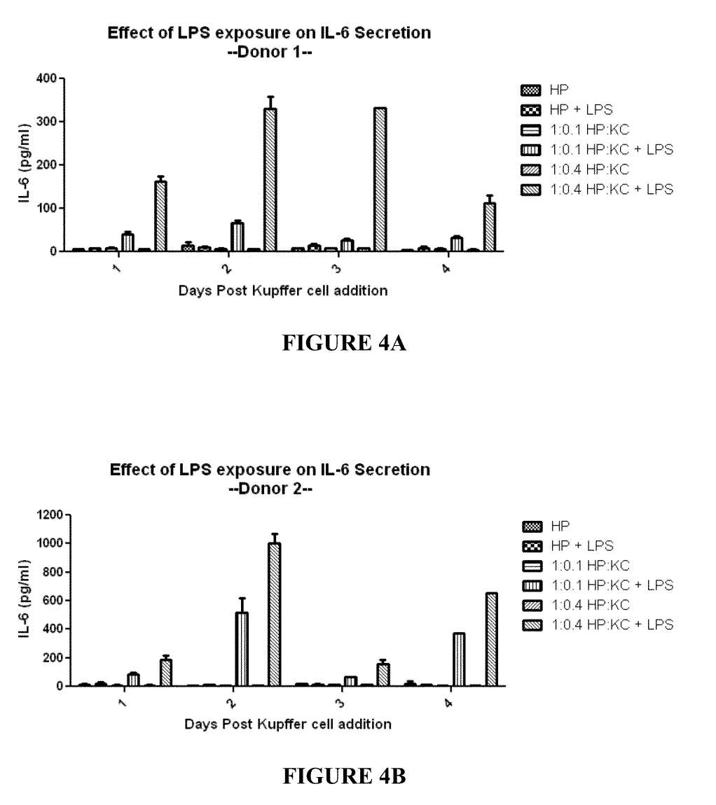

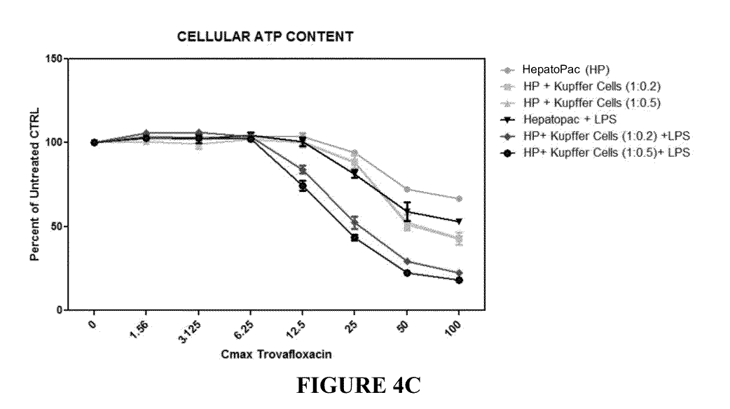

LPS stimulation of Kupffer cells causes release of IL-6. In addition, LPS stimulation of the HepatoPac™-Kupffer co-culture causes suppression of CYP3A4 activity potentially mediated by cytokines Co-cultures were stimulated overnight with 50 ng/mL of LPS at Day 1, 2, 3 and 4 post addition of Kupffer cells. Stimulation of this model with LPS caused secretion of IL-6 and TNF-α (not shown) for 4 days in culture at levels similar to those in LPS-stimulation of cultures of Kupffer cells alone indicating the presence of functional Kupffer cells. As shown in FIGS. 4A-4B, in both Kupffer cell donors tested, increasing IL-6 levels were observed with LPS stimulation and increasing Kupffer cell number. Furthermore, effect of LPS stimulation is Kupffer cell dependent as insignificant amounts of IL-6 were detected in Hepatocyte only co-cultures. FIG. 4C shows the effect of LPS stimulation on the cellular ATP content of Kupffer cells/HepatoPac™ co-cultures.

### Example 3

### Responsiveness to IL-1B

Cultures received 2 or 4 day treatment with IL-2, IL-1B, TNF-α, or IL-6 in serum-free media after which cytokine levels were assayed in their supernatants, Cyp3A4 activity was determined using the P450-Glo Assay (Promega), total ATP was measured (Promega Cell-Titer Glo) and RNA was extracted (Qiagen Rneasy). Taqman primer-probes from Life Technologies were used to determine relative gene expression changes verses the vehicle control via the ΔΔCt Method with significance confirmed via a two-tailed T-test.

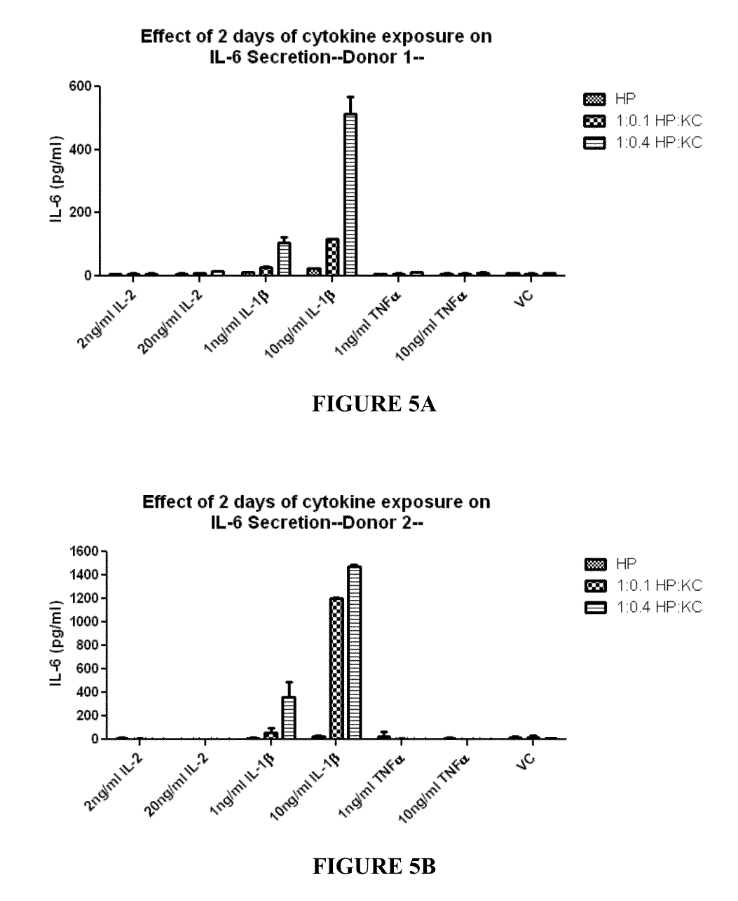

Co-cultures were exposed to IL-2, IL-1B and TNF-α and secretion of IL-6 into the culture supernatant was measured. FIGS. 5A-5B, show that exposure to IL-1B causes IL-6 release and is Kupffer cell enhanced as well as dose dependent. IL-2 and TNF-α had no effect on IL-6 secretion.

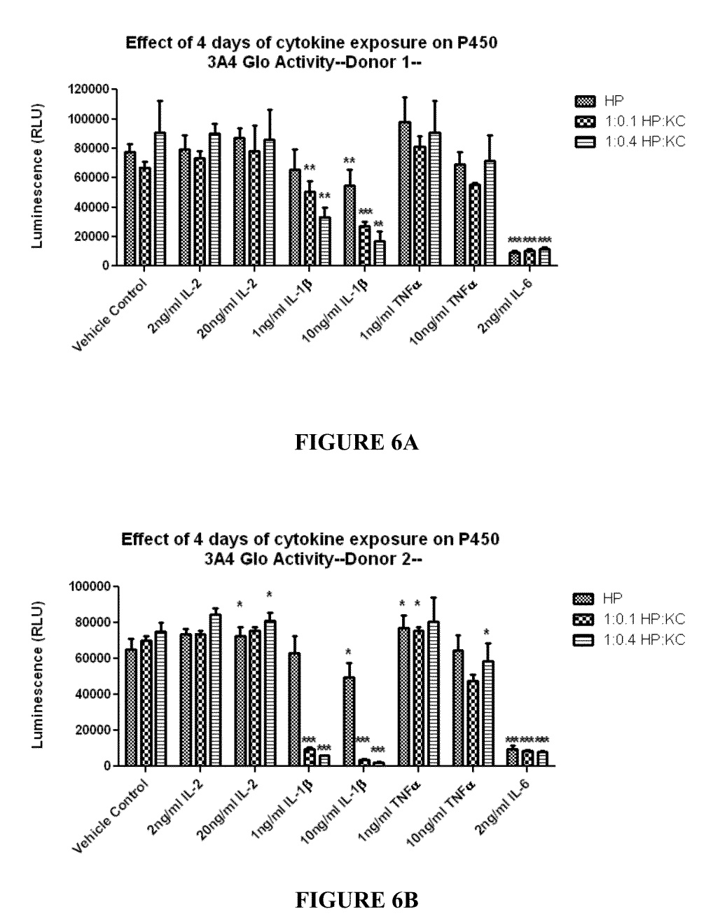

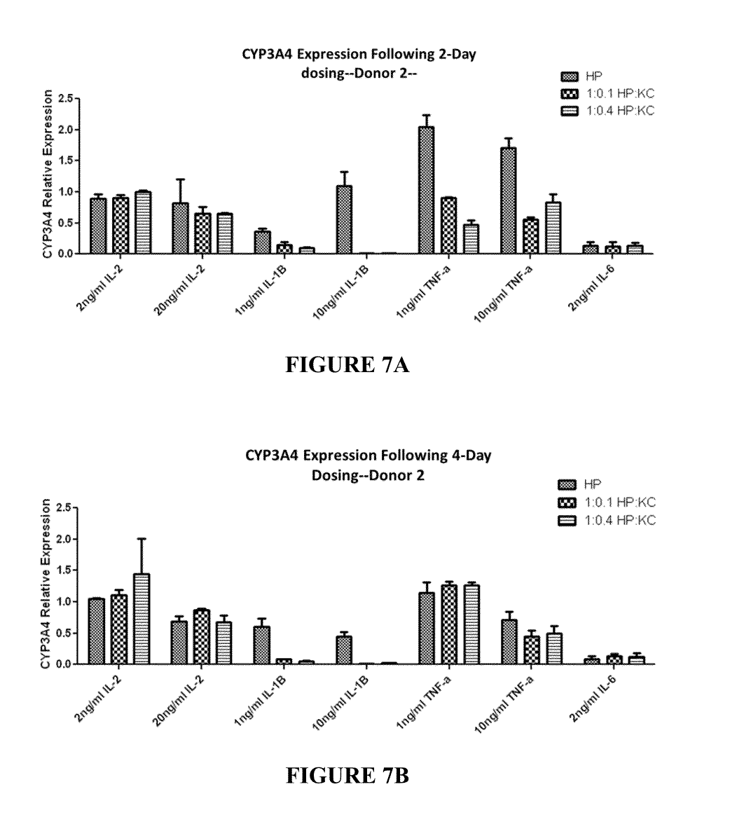

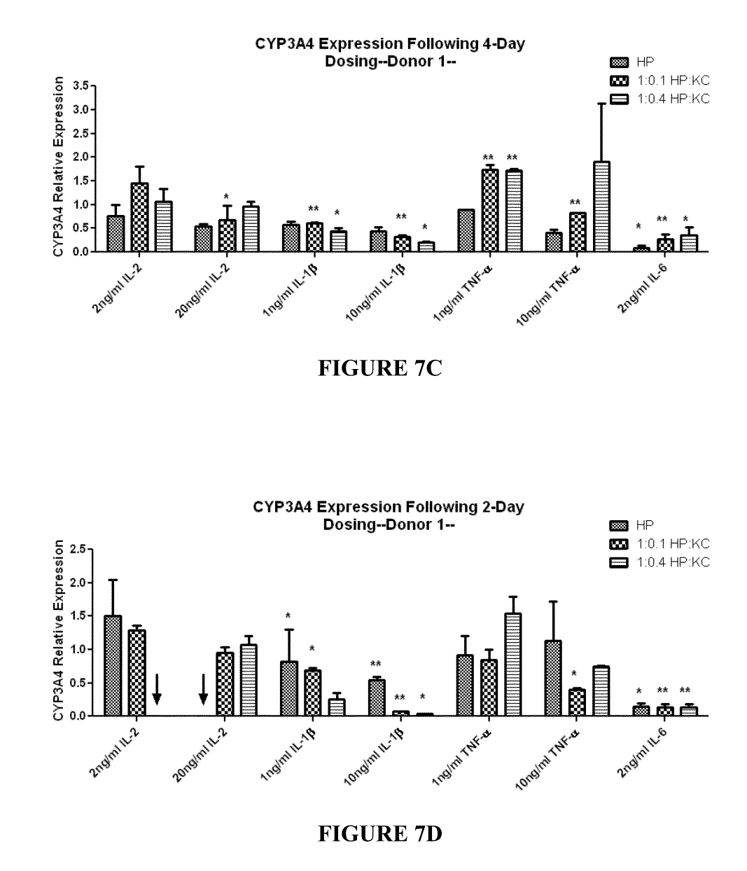

Repression of both Cyp3A4 activity and CYP3A4 expression were observed through the dose-dependent and Kupffer-cell enhanced effects of IL-1B. FIGS. 6A and 6B show cytokine-mediated Cyp450 3A4 inhibition. Treatment of tri-cultures with IL-1B demonstrates a dose-dependent and Kupffer cell-enhanced repression of Cyp3A4 activity which remains consistent over time. IL-6 treatment of cultures maintains repression of Cyp3A4 activity independent of Kupffer cells. FIGS. 7A-7D show cytokine-mediated repression of cytochrome expression. Repression of CYP3A4 expression occurs in an IL-1B dose-dependent and Kupffer cell-enhanced manner similar to protein activity inhibition shown in FIGS. 6A-6B. This same trend is evident in the repression of CYP2D6 as well (not shown).

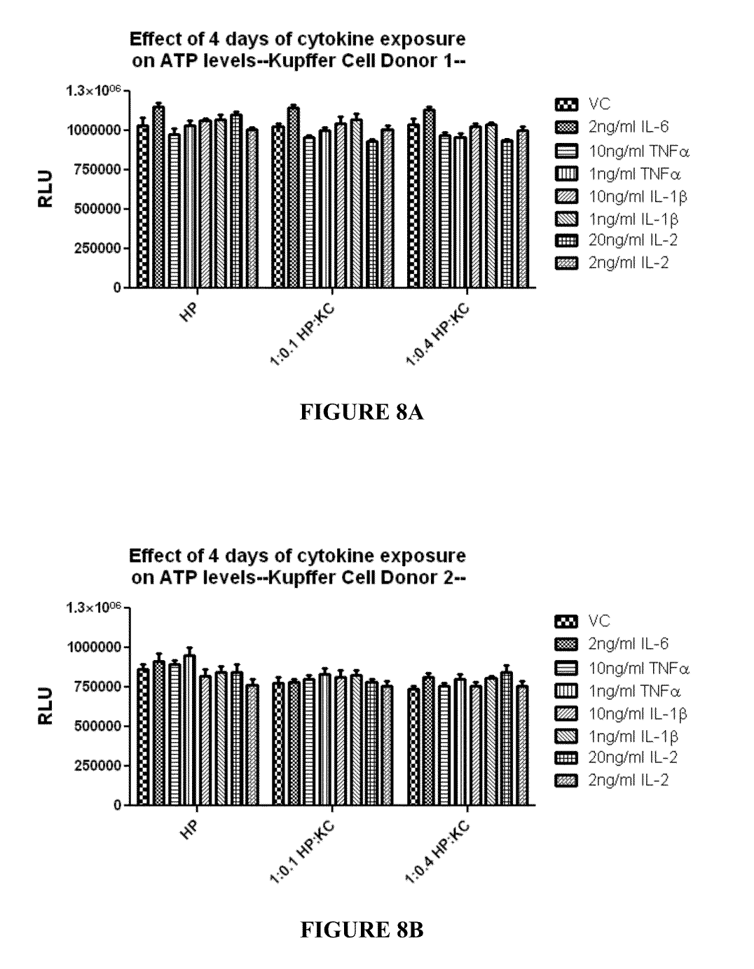

FIGS. 8A and 8B show the effect of cytokine exposure on cell viability. ATP levels were determined after exposure to cytokines for 4 days. As shown in FIGS. 8A and 8B, exposure to various concentrations of cytokines tested did not affect overall cell viability of the tri-cultures.

### Example 4

### In Vitro Model for Liver Inflammatory Modeling

It has been proposed that inflammatory stress may precipitate an idiosyncratic adverse drug reaction (IADR) in the liver such as that observed during the administration of the fluoroquinolone antibiotic trovafloxacin (TVX) (Tukov, Toxicol In Vitro. 20(8):1488-1499, 2006). Previous work performed by Shaw et al. in an in vivo mouse model demonstrated that LPS-induced inflammatory stress rendered TVX, but not its non-toxic analog levofloxacin (LVX), toxic. TNF-α was implicated as the pro-inflammatory mediator of this toxic response, an observation supported by toxicity abrogation in the presence of pentoxifylline and etanercept (Shaw et al. Toxicological Sciences. 100(1):259-266, 2007). Using TVX as a prototype compound, we evaluated the ability of the HepatoPac™-Kupffer cell co-culture model to detect inflammation-mediated toxicities.

HepatoPac™ co-cultures were first allowed to stabilize functionally in serum-supplemented media for a 7-day period. Species-matched Kupffer cells were added to human and rat HeaatoPac™ at multiple ratios (to mimic both the normal and inflamed state of the liver). To mimic the physiological state, human and rate Kupffer cells were added at hepatocyte:Kupffer cell ratios of 1:0.1 and 1:0.2, respectively. To mimic the inflamed state human Kupffer cells (at a ratio of 1:0.4 hepatocytes: Kupffer cells) or rat Kupffer cells (at a ratio of 1:0.5 hepatocytes: Kupffer cells) were then added to the cultures forming a triculture of fibroblasts, hepatocytes and kupffer cells. On day 8 of culture, the tricultures were treated with increasing concentrations of either Trovafloxacin or Levofloxacin (0-400 μM) in serum-free media. 24 hrs later, the tricultures were stimulated with 50 ng/mL LPS. HepatoPac™ alone cultures and HepatoPac™ cultures treated with LPS were used as controls in these experiments. Aliquots of the culture medium and cell lysates from each treatment group were collected for assessment of drug-induced effects on hepatocellular functions and cytotoxicity as described earlier. Trovafloxacin and levofloxacin were used as positive and negative control compounds.

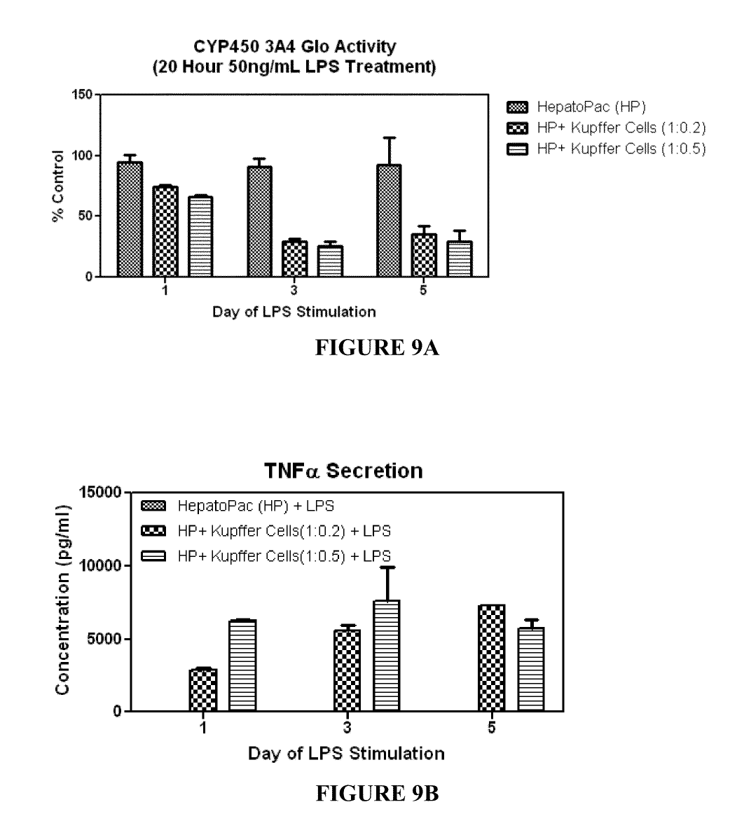

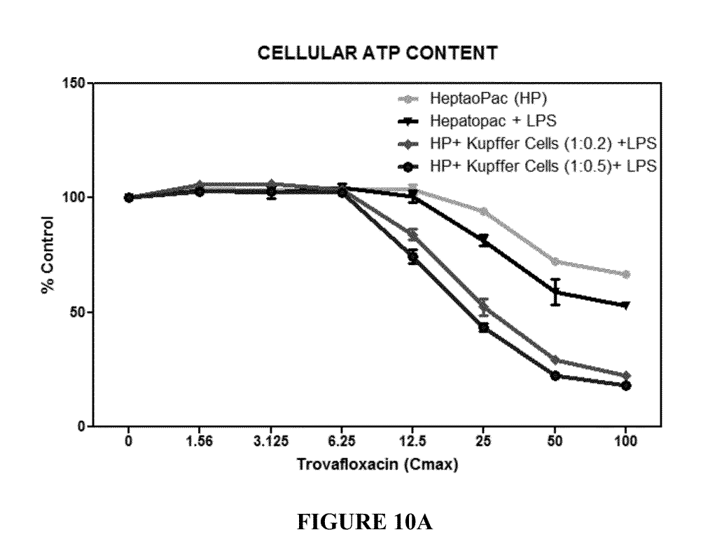

Stimulation of rat Kupffer-HepatoPac™ at different time points with 50 ng/mL LPS for 20 hours caused robust TNF-α secretion and downregulation of CYP3A4 activity. FIG. 9A is a graph of CYP450 3A4 Glo activity, which shows the variation in percent control at each of one, three and five days in culture after LPS stimulation. FIG. 9B shows TNF-α secretion, which is measure in concentration, in units pg/ml, at each of one, three and five days after LPS stimulation.

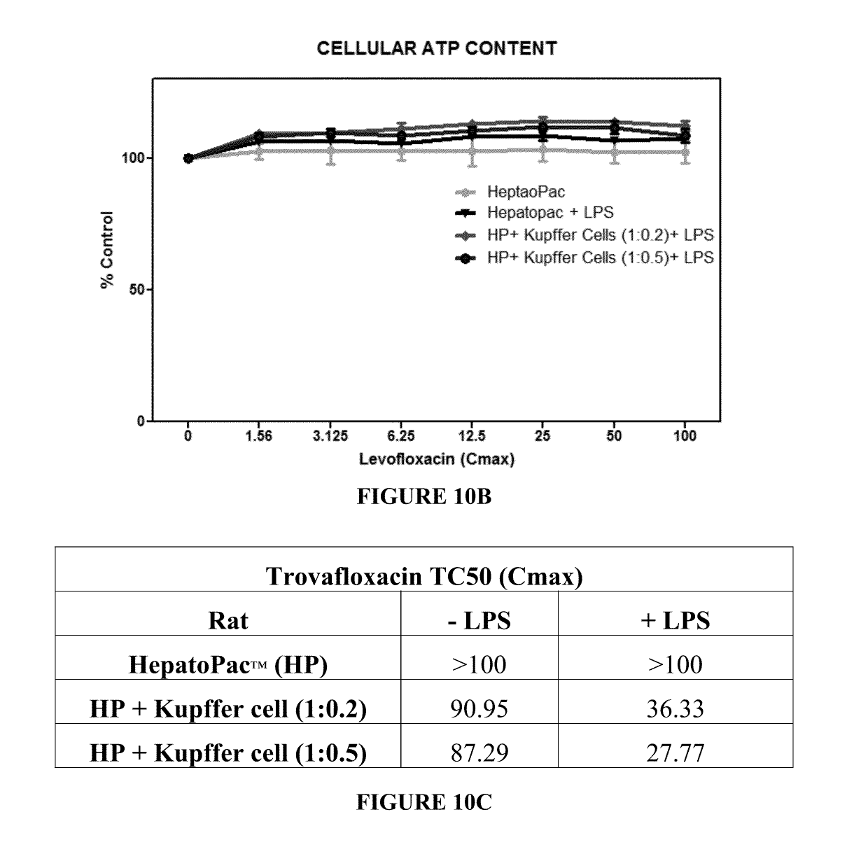

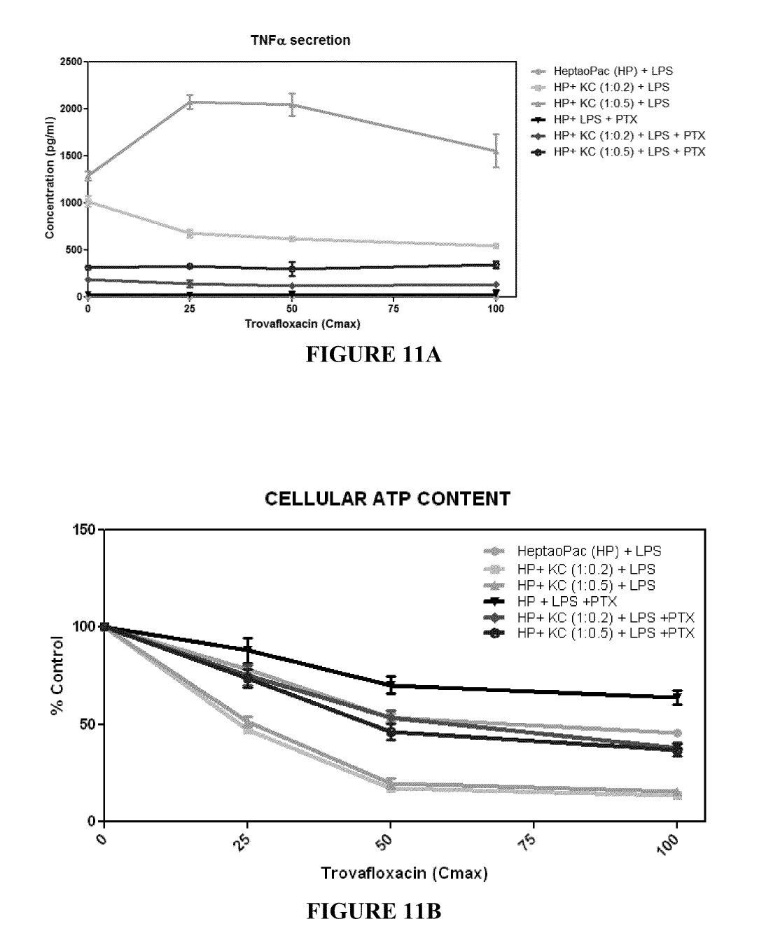

Trovafloxacin (TVX) toxicity is potentiated in LPS-treated rat HepatoPac™-kupffer cell co-cultures. FIG. 10A shows cellular ATP content of each of HepatoPac™, HepatoPac™ with LPS stimulation, HepatoPac™-Kupffer (1:0.2) cell co-cultures with LPS stimulation, and HepatoPac™-kupffer (1:0.5) cell co-cultures with LPS stimulation and illustrates variation in percent control versus Trovafloxacin (Cmax) for each. FIG. 10B shows cellular ATP content of each of HepatoPac™, HepatoPac™ with LPS stimulation, HepatoPac™-kupffer (1:0.2) cell co-cultures with LPS stimulation, and HepatoPac™-kupffer (1:0.5) cell co-cultures with LPS stimulation and illustrates the variation in the percent control versus Trovafloxacin (Cmax) for each. TVX showed characteristic dose-dependent cytotoxicity when added to rat HepatoPac™-Kupffer cell co-cultures. Stimulation of the cultures with LPS exacerbated TVX-induced toxicity as seen in FIG. 10A where there is a leftward shift (lower TC50 values) in the dose-response curves for ATP content. Levofloxacin was not toxic to rat HepatoPac™-Kupffer cell cultures even when stimulated with LPS.

Treatment with pentoxifylline (an inhibitor of TNF-α transcription) significantly decreased TVX/LPS-induced HepatoPac™ toxicity and TNF-α secretion, according to one embodiment. Cultures were treated with Trovafloxacin and 5 mM Pentoxifylline for 72 hours. After 24 hours of dosing, Kupffer cells were activated with 50 ng/ml of LPS.

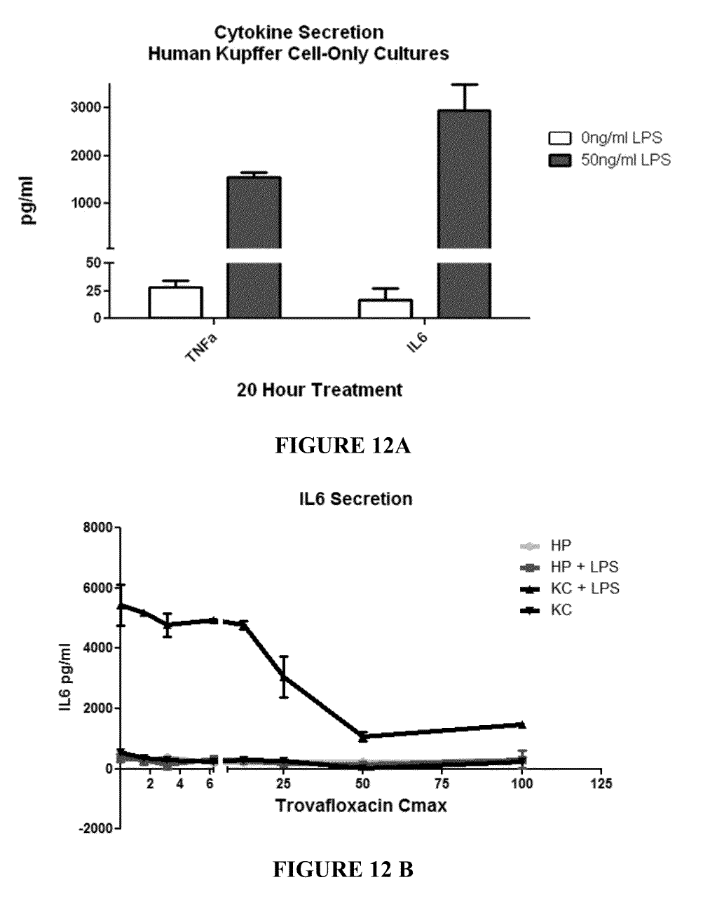

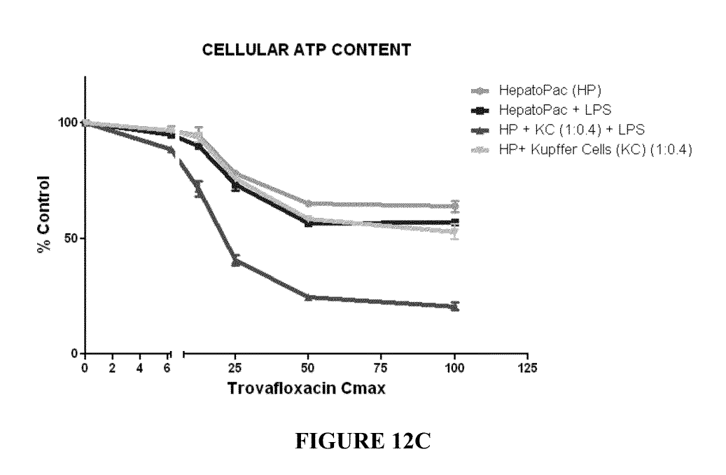

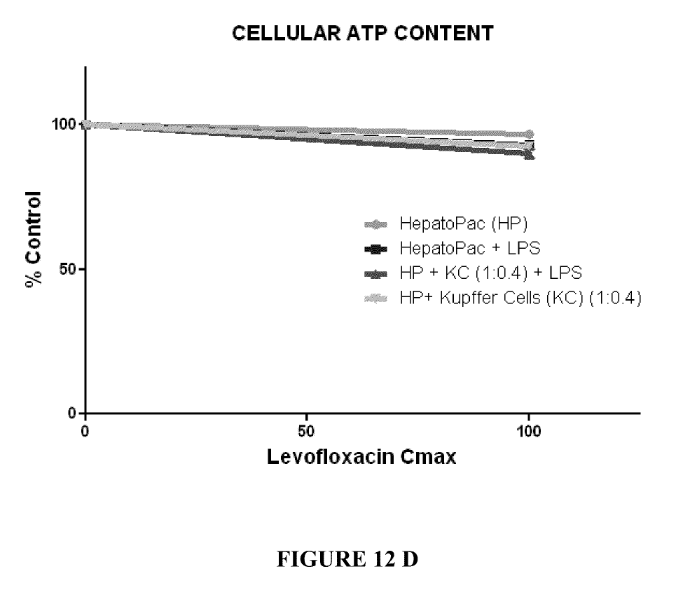

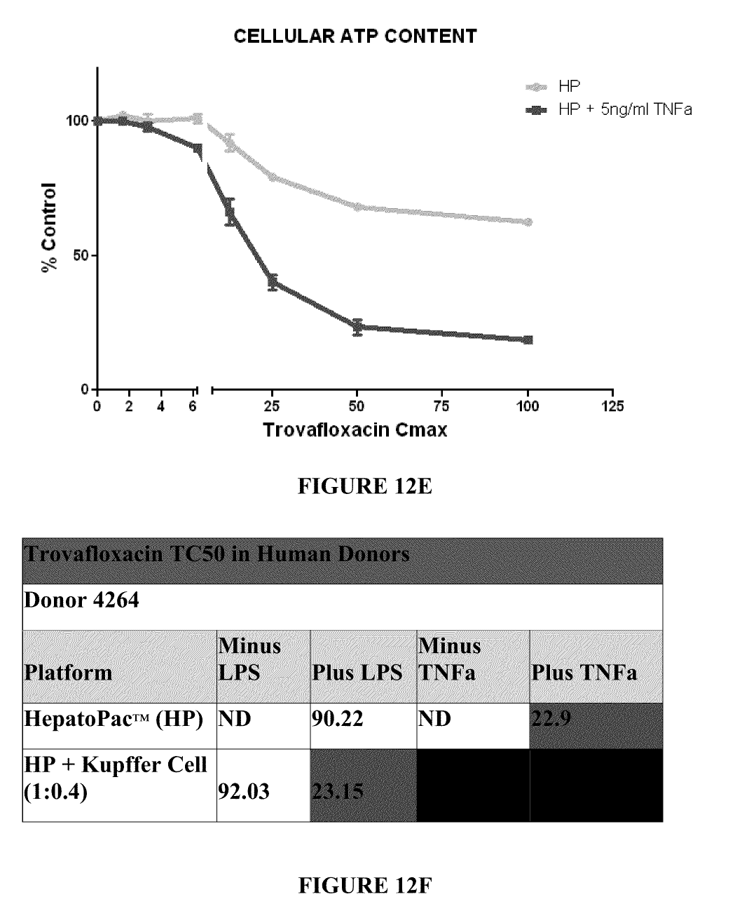

Trovafloxacin (TVX) toxicity is potentiated in LPS-treated human HepatoPac™-Kupffer cell co-cultures and independently by TNF-α. Stimulation of the co-cultures with LPS exacerbated TVX-induced toxicity as seen in FIG. 12C where there is a leftward shift (lower TC50 values) in the dose-response curves for ATP content. Additionally, addition of TNF-α to HepatoPac™ cultures potentiates TVX toxicity.

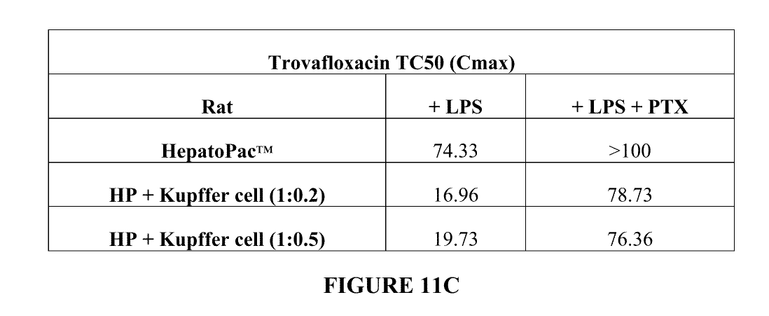

To summarize, we assessed whether stimulation of HepatoPac™-Kupffer cell co-cultures with LPS sensitizes the cultures to trovafloxacin (TVX) toxicity. Rat or human HepatoPac™-Kupffer cell co-cultures were treated with increasing concentrations of TVX (+/−LPS) and assessed for changes in hepatic ATP content. TVX caused a concentration dependent toxicity in the HepatoPac™-Kupffer cell co-cultures which was potentiated upon addition of 50 ng/mL LPS to the cultures (TC50=87.29 vs 27.77 Cmax for the rat platform and 92.03 vs 23.15 Cmax for the human platform) (FIGS. 11C and 12F). This effect was not observed with the non-toxic analog, levofloxacin. Treatment with pentoxifylline (an inhibitor of TNF-α transcription) significantly decreased TVX/LPS-induced rat HepatoPac™ toxicity suggesting a synergistic effect between TNF-α and trovafloxacin (TC50=19.73 vs. 76.36 Cmax). In conclusion, rat or human HepatoPac™-Kupffer cell co-cultures may be used to predict drug induced liver injury mediated by inflammatory stress.

While specific embodiments of the subject matter have been discussed, the above specification is illustrative and not restrictive. Many variations will become apparent to those skilled in the art upon review of this specification and are within the scope of the invention.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A micropatterned co-culture comprising:
(a) a population of hepatocytes defining a cellular island, wherein the cellular island comprises a diameter or width of about 250 μm to 750 μm;
(b) a population of stromal cells, wherein the stromal cells define a geometric border of the cellular island; and
(c) a population of Kupffer cells,
wherein the micropatterned co-culture of hepatocytes and Kupffer cells maintains long-term functional stability.

2. The micropatterned co-culture of claim 1, wherein the stromal cells are fibroblast cells or fibroblast derived cells.

3. The micropatterned co-culture of claim 1, further comprising one or more populations of non-parenchymal cells.

4. The micropatterned co-culture of claim 3, wherein the one or more populations of non-parenchymal cells are selected from the group consisting of Ito cells, endothelial cells, biliary duct cells, immune-mediating cells, and stem cells.

5. The micropatterned co-culture of claim 4, wherein the immune-mediating cells are selected from the group consisting of macrophages, T cells, neutrophils, dendritic cells, mast cells, eosinophils and basophils.

6. The micropatterned co-culture of claim 1, wherein the ratio of hepatocytes to Kupffer cells in the micro-patterned co-culture is 1:0.1.

7. The micropatterned co-culture of claim 1, wherein the ratio of hepatocytes to Kupffer cells in the micro-patterned co-culture is 1:0.4.

8. The micropatterned co-culture of claim 1, wherein the cellular islands are spaced apart from about 1200 μm to 1300 μm from center to center of the cellular islands.

9. The micropatterned co-culture of claim 1, located in a microfluidic device.

10. The micropatterned co-culture of claim 1, located in a tissue culture plate.

11. The micropatterned co-culture of claim 1, wherein the micropatterned co-culture of hepatocytes and Kupffer cells maintains long-term functional stability for at least 10 days.

12. The micropatterned co-culture of claim 1, wherein the hepatocytes and Kupffer cells are selected from the group consisting of human cells, rat cells, mouse cells, monkey cells, dog cells, fish cells and guinea pig cells.

13. A method for producing a micropatterned co-culture containing at least three cell types, the method comprising:
(a) spotting an adherence material on a substrate at spatially different locations, each spot having a defined geometric pattern, wherein the defined geometric pattern comprises a diameter or width of about 250 μm to 750 μm;
(b) contacting the substrate with a population of hepatocytes that selectively adhere to the adherence material and/or substrate;
(c) culturing the hepatocytes on the substrates to generate a plurality of cellular islands; and
(d) contacting the substrate with a stromal cell population that adheres to the substrate at a location different than the hepatocyte population, wherein the cells of the stromal cell population define a geometric border of the cellular island, to generate a hepatocyte-stromal cell co-culture;
(e) maintaining the hepatocyte-stromal cell co-culture for a period of time sufficient to allow the hepatocytes to functionally stabilize; and
(f) contacting the hepatocytes and stromal cells with a population of Kupffer cells;
wherein the micropatterned co-culture of hepatocytes and the Kupffer cells maintains long-term functional stability.

14. The method of claim 13, wherein period of time sufficient to allow the hepatocytes to functionally stabilize is at least 7 days.

15. The method of claim 13, wherein functional stability of the heptocytes is determined by measuring an activity selected from gene expression, cell function, metabolic activity, morphology, and a combination thereof, of the hepatocytes.

16. The method of claim 15, wherein the metabolic activity is selected from CYP3A4 activity, urea synthesis, and albumin secretion.

17. A cellular composition made by the method of claim 13.

18. A method of determining the interaction of one or more test compounds with hepatocytes comprising:
a) contacting a micropatterned co-culture comprising hepatocytes, stromal cells, and Kupffer cells with one or more test agents; and
b) measuring a characteristic of the one or more test compounds or an activity of the hepatocytes, wherein the characteristic or activity measured in (b) indicates the interaction of one or more test compounds with hepatocytes and wherein the micropatterned co-culture of hepatocytes and Kupffer cells maintains long-term functional stability.

19. A method of determining the effect of liver inflammation on one or more test compounds comprising:
a) contacting a micropatterned co-culture comprising hepatocytes, stromal cells, and Kupffer cells with an inflammation-inducing agent to generate an in vitro model of liver inflammation;
b) contacting the in vitro model of liver inflammation generated in step (a) with one or more test agents; and
c) measuring a characteristic of the one or more test compounds or an activity of the hepatocytes, wherein the characteristic or activity measured in (c) indicates the effect of liver inflammation on the one or more test compounds.

20. The method of claim 18 or 19, wherein the activity of the hepatocytes is selected from gene expression, cell function, metabolic activity, morphology, cytokine secretion, protein or metabolite secretion, and a combination thereof.

21. The method of claim 18 or 19, wherein the micropatterned co-culture of hepatocytes and Kupffer cells maintains long-term functional stability for at least 10 days.

22. The method of claim 18 or 19, wherein the ratio of the Kupffer cells to the hepatocytes corresponds to the ratio of the cells in an inflamed state of the liver.

23. The method of claim 18 or 19, wherein the ratio of the Kupffer cells to the hepatocytes corresponds to the ratio of the cells in a physiologically normal state of the liver.

24. The method of claim 18 or 19, wherein the test agent is selected from the group consisting of a cytotoxic agent, pharmaceutical agent, a small molecule, and a xenobiotic.

25. The method of claim 18 or 19, wherein the metabolic activity is protein production.

26. The method of claim 18 or 19, wherein the metabolic activity is enzyme bioproduct formation.

27. The method of claim 18 or 19, wherein the metabolic activity is a CYP450 isoenzyme activity.

28. The method of claim 27, wherein the CYP450 isoenzyme is selected from the group consisting of CYP1A2, CYP1B1, CYP2A6, CYP2B6, CYP2C, CYP2D6, CYP2E1, CYP2F1, CYP2J2, CYP3A4, CYP4A, and CYP4B.

29. The method of claim 19 for use in determining inflammation-mediated toxicity of a test agent.

30. The method of claim 18 or 19 for use in determining inflammation-mediated effects on co-administered test agent combinations.

31. The method of claim 18 or 19, wherein a characteristic of the one or more test compounds is selected from mass, structure, quantity and a combination thereof.

32. The method of claim 19, further comprising:
d) contacting a second micropatterned co-culture comprising hepatocytes, stromal cells, and Kupffer cells with the one or more test agents;
e) measuring the activity selected in (b) of the second co-culture hepatocytes; and
f) comparing the measurements in step (b) and (e).

33. A method of determining inflammation-mediated toxicity of a test agent, comprising;
a) contacting a first micropatterned co-culture comprising hepatocytes, stromal cells, and Kupffer cells with the test agent;
b) measuring an activity selected from gene expression, cell function, metabolic activity, morphology, and a combination thereof, of the hepatocytes;
c) contacting a second micropatterned co-culture comprising hepatocytes, stromal cells, and Kupffer cells with an inflammation-inducing agent to generate an in vitro model of liver inflammation;
d) contacting the in vitro model of liver inflammation generated in step (c) with the test agent;
e) measuring the activity selected in (b) of the second co-culture hepatocytes; and
f) comparing the measurements in step (b) and (e), to determine the inflammation-mediated toxicity of the test agent.

34. The method of claim 33, wherein the inflammation-inducing agent is LPS

35. The method of claim 33, wherein the inflammation-inducing agent is IL-1B.

36. The method of claim 33, wherein the inflammation-inducing agent is selected from the group consisting of a cytotoxic agent, pharmaceutical agent, and a xenobiotic.

37. The method of claim 33, wherein the test agent is selected from the group consisting of a cytotoxic agent, pharmaceutical agent, a small molecule, and a xenobiotic.
